(19) United States
(12) Patent Application Publication
(10) Pub. No.: US 2011/0117020 A1
(43) Pub. Date: May 19, 2011

(54) IMAGING DENDRIMER NANOPROBES AND USES THEREOF

(75) Inventors: Sergei A. Vinogradov, Wynewood, PA (US); Artem Y. Lebedev, Culveo City, CA (US)

(73) Assignee: THE TRUSTEES OF THE UNIVERSITY OF PENNSYLVANIA, Philadelphia, PA (US)

(21) Appl. No.: 12/919,647

(22) PCT Filed: Mar. 2, 2009
(86) PCT No.: PCT/US2009/035766

§ 371 Date: Feb. 7, 2011

Related U.S. Application Data
(60) Provisional application No. 61/064,329, filed on Feb. 28, 2008.

(51) Int. Cl. A61K 49/00; A61B 5/05

(52) U.S. Cl. 424/9.1; 600/407

(57) ABSTRACT

This invention relates to imaging nanoprobe and methods of use thereof. Specifically, the invention relates to long-lived oxygen-insensitive nanoprobe comprising a lumisescent moeity with a long excited state lifetime, embedded in a dendrimer, wherein said dendrimer is internally cross-linked and has hydrophilic peripheral layer.

### GOVERNMENT INTEREST

This invention was supported, in part, by Grant Numbers RO1-EB007279, HL081273, and NS031465 from the National Institutes of Health. The government may have certain rights in the invention.

### FIELD OF INVENTION

This invention relates to imaging nanoprobe and methods of use thereof. Specifically, the invention relates to nanoprobe comprising a lumisescent moeity with long excited state lifetime (microseconds and more) embedded in a dendrimer, where the said dendrimer is able to isolate the moiety from unwanted contacts with the environment and entirely protect it from unwanted quenching by oxygen.

### BACKGROUND OF THE INVENTION

Luminescent labels (or probes) are used extensively in analytical techniques, including multiple applications in biology and medicine. In theory, sensitivity of luminescence-based detection is limited only by photon counting capability, and can be, in principle, taken up to the single-molecule level. However, in practice Signal-to-Noise Ratios (SNR) are greatly reduced due to a number of instrument-, sample- or probe-related reasons. These include, but not limited by, auto-luminescence—a contaminant signal occurring in the same wavelength interval as the signal of interest; scattering, which deflects both excitation and emission photons, diminishing the detection efficiency; unwanted quenching of luminescence by various small molecule quenchers, e.g. water, oxygen or electroactive molecules. In relation to these issues, the following characteristics of luminescent probes should be considered:
1. Brightness is the key to the detection capability. Brightness is defined as a product of the molar extinction coefficient and the emission quantum yield;
2. Large Stokes shift provides efficient spectral separation between the emission and excitation photons;
3. Near-Infrared (NIR) excitation and emission bands are preferred over UV or visible bands, since minimally overlap with endogenous absorption and auto-luminescence of biological molecules;
4. Long luminescence lifetimes provide means for time-gating—the most efficient way to eliminate background signals. Autofluorescence usually decays after about 20 ns. If the lifetime of the probe/label is considerably longer (e.g. in many microsecond range), time-gated detection makes it possible to increase the SNR by orders of magnitude;
5. Lack of unwanted quenching is critical for the probe performance. Molecules in their excited states are typically much more reactive than in the ground states. As a result, luminescence quantum yields, especially of long-lived probes, are strongly diminished in the presence of e.g. oxygen, water and/or various electroactive quenchers. Therefore, long-lived probes protected from small-molecule quenchers will offer great advantages over quenchable long-lived probes and/or non-quenchable but short-lived (fluorescent) probes;

The remaining requirements are derived from the demands of biological systems:
6. Low toxicity, which typically means low chemical reactivity, e.g. with respect to biological substrates; and
7. High water-solubility and/or no aggregation in aqueous environments. Although apparently simple, this requirement is very difficult to meet in practice. Organic chromophores are usually intrinsically hydrophobic, whereas their modification with polar hydrophilic groups—the most common way to increase water-solubility, often leads to significant perturbations of their optical properties. Methods of building hydrophilic non-polar dyes with optical properties close to those of the original hydrophobic chromophores need to be developed.

Extensive research has been conducted over the years in an effort to create better luminescent probes and improve methods of luminescence detection. Long-lived probes attract special attention since they permit time-gated detection and, therefore, lead to much higher SNR's.

Currently existing long-lived probes have a number of drawbacks limiting their practicality. For example, lanthanide-based probes are sensitive to pH, exhibit low brightness and cannot be excited in the NIR region. Phosphorescent metalloporphyrins, e.g. Pt and Pd porphyrins and their derivatives, are extremely sensitive to oxygen, and while being very bright in deoxygenated environments show practically no emission at ambient oxygen concentrations. Ruthenium and related complexes are less sensitive to oxygen because of their relatively short lifetimes (microseconds as opposed to tens and hundreds of microseconds in the case of metalloporphyrins), but are not always stable in aqueous environments and exhibit moderate brightness. In addition, their short lifetimes diminish time-gating capability.

Several approaches have been proposed in the past to overcome these problems. For example, lanthanide ions have been imbedded into polymeric nano-particles in order to prevent their contacts with water. Unfortunately, the resulting probes have excessively large sizes, impeding functions of biological analytes, e.g. antibodies. Metalloporphyrin-based labels have been used in combination with oxygen scavengers. Although certain increase in their phosphorescence quantum yields has been achieved, chemical depletion of oxygen is cumbersome and incompatible with many types of biological systems.

### SUMMARY OF THE INVENTION

In one embodiment, this invention provides a quencher-insensitive nanoprobe comprising: a lumisescent moeity with a long excited state lifetime (microseconds and more) embedded in a dendrimer, having a hydrophilic peripheral layer, wherein emission lifetime of the luminescent moiety is longer than the diffusion time of the quencher to the luminescent core.

In another embodiment, the invention provides a quencher-insensitive nanoprobe comprising: a lumisescent moeity with a long excited state lifetime, embedded in a dendrimer, wherein emission lifetime of the luminescent moiety is longer than the diffusion time of the quencher to the luminescent core.

In one embodiment, the invention provides a method for an in-vivo imaging of a tumor neovasculature in an individual comprising (i) administering a quencher-insensitive nanoprobe comprising: a lumisescent moeity with a long excited state lifetime, embedded in a dendrimer, optionally having a hydrophilic peripheral layer, wherein emission lifetime of the luminescent moiety is longer than the diffusion time of the quencher to the luminescent core; (ii) exciting said luminescent moeity; (iii) detecting light emitted from said tumor neovasculature.

In another embodiment, the invention provides an optical imaging system comprising: an electronic imaging device configured to capture an image of a predetermined site; a nanoprobe comprising: a lumisescent moeity with a long excited state lifetime, embedded in a dendrimer with hydrophilic peripheral layer, wherein said dendrimer isolates the chromophore from the measurement environment and eliminates unwanted quenching by posing a kinetic barrier to the quenching species; and a projector configured to project a visible representation of the captured image.

In another embodiment, the nanoprobe is presented by the general formula: C-(Dⁿ-R)ₘ, where is a dendritic core, D is a dendritic skeleton, n is a generation number, R is the polymeric unit which optionally is linked to a moiety specific to the pre-determined target and m is the number of dendritic wedges attached to the core.

In another embodiment the nanoprobe is presented by the general formula: C-(D_{c}ⁿ-R)ₘ, where is a dendritic core, D_{c} is a cross-linked dendritic skeleton, n is a generation number, R is the polymeric unit which optionally is linked to a moiety specific to the pre-determined target and m is the number of to dendritic wedges attached to the core.

Other features and advantages of the present invention will become apparent from the following detailed description examples and figures. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

The invention will be better understood from a reading of the following detailed description taken in conjunction with the drawings in which like reference designators are used to designate like elements, and in which:

FIG. 1 shows a general scheme of dendritically protected long-lived non-quenchable luminescent probe.

FIG. 2 shows Metalloporphyrin-based long-lived luminescent probe protected by G2 Aryl-Glycine (AG) dendrimer.

FIG. 3 shows selected absorption and emission spectra of metalloporphyrins.

FIG. 4 shows a G3 AG-dendron.

FIG. 5 shows dendritically protected phosphorescent probe with single attachment site: a) dendrimerization procedure; b) attaching to a specific antibody.

FIG. 6 shows dendritic phosphorescent probe with specific surface area suitable for reaction with biological analytes: a) initial dendrimerization procedure; b) attaching dendron with other terminal protection groups, followed by deprotection; c) attachment to the analyte.

FIG. 7 shows construction of a cross-linked dendritic matrix A—reactive groups, activated upon action of radiation, heat or some initiating reagent.

FIG. 8 shows three basic structural types of porphyrins used as sensing elements of phosphorescent probes. (PEG refers to monomethoxyoligoethylene glycol, Av. MW 350). Absorption and emission spectra complexes Pd-1-OBu, Pd-2-OBu and Pd-3-OBu.

FIG. 9 shows X-ray crystal structure of Pt tetraaryltetranaphthoporphyrin Pt-3-Bu.

FIG. 10 shows absorption (a) and emission (b) spectra of the G2 dendrimers based on Pt porphyrins with increasing degree of π-conjugation. The emission spectra are scaled to reflect the relative phosphorescence quantum yields in the absence of quenching.

FIG. 11 shows oxygen quenching plots for A) Pt-1-OPEG, B) Pt-1-AG¹-OPEG (8), C) Pt-1-AG²-OPEG (9), D) Pt-1-AG³-OPEG (11), E) Pt-2-AG²-OPEG (12), F) Pt-2-AG²-OPEG (13) and G) Pt-3-AG²-OPEG (14).

FIG. 12 shows scheme 1, Synthesis of Pd and Pt complexes of TBP's and TNP's. TNP's and the corresponding precursors are shown with dashed lines. i) modified Barton-Zard reaction with ethylisocyanoacetate; ii) hydrolysis/decarboxylation; iii) protection of the aldehyde with 1,3-propanediol; iv) Pd(0)-catalyzed butoxycarbonylation; v) deprotection by THF/HCl; yl) Lindsey condensation; vii) metal insertion; viii) oxidative aromatization with DDQ; xi) base-mediated hydrolysis.

FIG. 13 shows Scheme 2, Synthesis of poly(aryl-glycine) (AG) dendrons. CDMT—2-chloro-4,6-dimethoxy-1,3,5-triazine; NMM—N-methylmorpholine; HBTU—o-benzotriazole-N,N,N′,N′-tetramethyluronium hexafluorophosphate; DIPEA—N,N-Diisopropylethylamine.

FIG. 14 shows dendrimer synthesis and modification. In the first line, C (core) designates porphyrin-octacarboxylic acids: Pt-1-OH, Pd-1-OH, Pt-2-OH, Pd-2-OH, Pd-3-OH. The table shows the numbering scheme for the probes of the general formula C-(AG²OPEG)₈) (See FIG. 2). HOBt—N-hydroxybenzotriazole; PEG—oligoethyleneglycol monomethylether Av. MW 350.

### DETAILED DESCRIPTION OF THE INVENTION

In some embodiment, this invention relates to an imaging nanoprobe comprising: a lumisescent moeity with a long excited state lifetime, embedded in a dendrimer with hydrophilic peripheral layer, wherein said dendrimer isolates the chromophore from the measurement environment and eliminates unwanted quenching by posing a kinetic barrier to the quenching species

In another embodiment, this invention provides a nanoprobe comprising an oxygen sensitive lumisescent moeity embedded in a dendrimer with hydrophilic peripheral layer

The luminescent moiety, or luminophore, is characterized in one embodiment, by a number of parameters, including extinction coefficient, quantum yield, and luminescence lifetime. In one embodiment, the term “Extinction coefficient” refers to the wavelength-dependent measure of the absorbing power of a luminophore. In another embodiment, the term “Quantum yield” refers to the ratio of the number of photons emitted to the number of photons absorbed by a luminophore. In another embodiment, the term “Luminescence lifetime” refers to the average time between absorption and re-emission of light by a luminophore. In one embodiment, Lanthanide luminescence is exceptional for its long luminescence lifetimes, which often are in the microsecond to millisecond range. In one embodiment, the term “long-lived excited state” refers to an excited state with lifetime longer than about 100 nanoseconds in the absence of quenching. In one embodiment, the nanoprobes described herein, which are used in the methods provided herein, have a luminescent moiety that emits from a long-lived excited state. In one embodiment, excited state lifetime is in the order of microseconds or longer. In another embodiment, the luminescent moiety is a luminescent transition metal complex, or a luminescent metalloporphyrin, or a complex of a luminescent lanthanide ion in other certain discrete embodiments of the luminescent moieties used in the nanoprobes described herein.

In one embodiment, the nanoprobes described herein, are assembled from three key building blocks: luminescent moeity, dendron and a polymeric unit. In another embodiment, the luminescent moeity, the dendron and the polymeric unit or layer are covalently attached. In another embodiment, the three key building block are linked via covalent bonds, non-covalent bonds or their combination.

In one embodiment, provided herein is a quencher-insensitive nanoprobe comprising: a lumisescent moeity with a long excited state lifetime in the order of 100's of nanoseconds, or microseconds or longer, embedded in a dendrimer, having a hydrophilic peripheral layer, wherein emission lifetime of the luminescent moiety is longer than the diffusion time of the quencher to the luminescent core.

In another embodiment, provided herein is a quencher-insensitive nanoprobe comprising: a lumisescent moeity with a long excited state lifetime, embedded in a dendrimer having a hydrophilic peripheral terminating group, such as hydroxyl in one embodiment and wherein emission lifetime of the luminescent moiety is longer than the diffusion time of the quencher to the luminescent core.

In one embodiment, provided herein is a method for in vivo imaging vasculature in an individual comprising (i) administering a nanoprobe comprising lumisescent moeity embedded in a dendrimer with hydrophilic peripheral layer; (ii) exciting said luminescent moeity by directing light; and (iii) detecting light emitted from said vasculature.

In one embodiment, provided herein is an optical imaging system comprising: an electronic imaging device configured to capture an image of a predetermined site; a nanoprobe comprising: a lumisescent moeity with a long excited state lifetime in the order of at least 100 nanosecond in one embodiment, embedded in a dendrimer with hydrophilic peripheral layer and a projector configured to project a visible representation of the captured image.

In another embodiment the luminescent moeity is a metalloporphyrin, the dendron is an aryl-glycine skeleton and the polymeric peripheral layer is polyethylene glycole unit. In another embodiment other long-lived luminescent dyes can be used, such as Ru in one embodiment, or Os, Ir complexes with bipyridyl and similar ligands in other discrete embodiment, to which protective dendrons are used. A person skilled in the art would readily recognize that dendrons are not limited to aryl-glycines, and in certain embodiments of the dendrons used in the nanoprobes described herein, any hydrophobic dendrimers foldable in aqueous environments can be used. In one embodiment, dendrimers consisting of aromatic motifs fold more tightly in aqueous solutions, thus offering an embodiment of an effective protection to embedded metalloporphyrins.

In another embodiment, the luminescent moiety is a phosphorescent metalloporphyrin with excitation bands throughout the UV-Vis-near infrared (NIR) range.

In one embodiment, the nanoprobe has a luminescent moiety that is a metalloporphyrin described by the compound by Formula I:

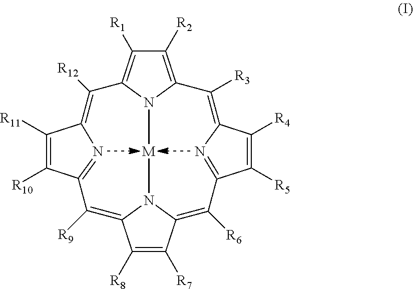

whereby M is a group 8 transition metal ion or a lanthanide metal ion;

R₁₋₁₂ are, independently, hydrogen, a substituted or unsubstituted alkyl, substituted or unsubstituted alkylene, substituted or unsubstituted hydroxyalkyl, substituted or unsubstituted haloalkyl, substituted or unsubstituted alkanoyl, substituted or unsubstituted allyl, substituted or unsubstituted haloalkyl, substituted or unsubstituted silylalkyl, substituted or unsubstituted cycloalkyl, substituted or unsubstituted aryl, amine, carboxylic acid, hydroxyl, azide, cyano,
—C(O)—O-alkyl or O—C(O)-alkyl. In another embodiment, M is a palladium ion. In another embodiment to M is a platinum ion. In another embodiment M is a ruthenium (Ru) ion.

In one embodiment, provided herein the imaging nanoprobe which is used in the methods provided herein, have a luminescent moiety that is a transition metal complex described by the compound by Formula II:

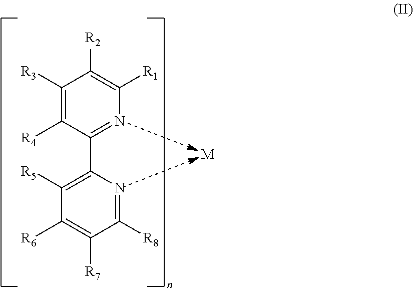

where, M is a group 8 transition metal ion, or a lanthanide ion;

R₁₋₈ are, independently, independently, hydrogen, a substituted or unsubstituted alkyl, substituted or unsubstituted alkylene, substituted or unsubstituted hydroxyalkyl, substituted or unsubstituted haloalkyl, substituted or unsubstituted alkanoyl, substituted or unsubstituted allyl, substituted or unsubstituted haloalkyl, substituted or unsubstituted silylalkyl, substituted or unsubstituted cycloalkyl, substituted or unsubstituted aryl, amine, carboxylic acid, hydroxyl, azide, cyano, —C(O)—O-alkyl or O—C(O)-alkyl; n=1, 2, 3 or 4.

In one embodiment, the nanoprobe which is used in the methods provided herein, has a luminescent moiety that is a lanthanide ion described by the compound by Formula 3:

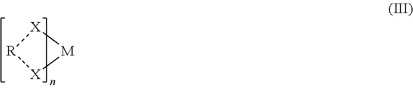

whereby M is any lanthanide metal atom;

X is, independently, nitrogen (N), oxygen (O), sulfur (S) or phosphorous (P) atom; R is a substituted or to unsubstituted alkyl, substituted or unsubstituted alkylene, substituted or unsubstituted hydroxyalkyl, substituted or unsubstituted haloalkyl, substituted or unsubstituted alkanoyl, substituted or unsubstituted allyl, substituted or unsubstituted haloalkyl, substituted or unsubstituted silylalkyl, substituted or unsubstituted cycloalkyl, substituted or unsubstituted aryl, amine, carboxylic acid, hydroxyl, azide, cyano, —C(O)—O-alkyl or O—C(O)-alkyl; n=1, 2, 3 or 4.

The term “cycloalkyl” refers to a non-aromatic, monocyclic or polycyclic ring comprising carbon and hydrogen atoms. A cycloalkyl group can have one or more carbon-carbon double bonds in the ring so long as the ring is not rendered aromatic by their presence. Examples of cycloalkyl groups include, but are not limited to, (C3-C7) cycloalkyl groups, such as cyclopropyl, cyclobutyl, cyclopentyl, cyclohexyl, and cycloheptyl, and saturated cyclic and bicyclic terpenes and (C3-C7) cycloalkenyl groups, such as cyclopropenyl, cyclobutenyl, cyclopentenyl, cyclohexenyl, and cycloheptenyl, and unsaturated cyclic and bicyclic terpenes. A cycloalkyl group can be unsubstituted or substituted by one or two substituents. Such substituents include a halogen, hydroxyl, amine, cyano, aldehyde, carboxylic acid or nitro group. Preferably, the cycloalkyl group is a monocyclic ring or bicyclic ring.

The term “alkyl” refers, in, one embodiment, to a saturated aliphatic hydrocarbon, including straight-chain, branched-chain and cyclic alkyl groups. In one embodiment, the alkyl group has 1-12 carbons. In another embodiment, the alkyl group has 1-7 carbons. In another embodiment, the alkyl group has 1-6 carbons. In another embodiment, the alkyl group has 1-4 carbons. In another embodiment, the cyclic alkyl group has 3-8 carbons. In another embodiment, the cyclic alkyl group has 3-12 carbons. In another embodiment, the branched alkyl is an alkyl substituted by alkyl side chains of 1 to 5 carbons. In another embodiment, the branched alkyl is an alkyl substituted by haloalkyl side chains of 1 to 5 carbons. The alkyl group may be unsubstituted or substituted by a halogen, haloalkyl, hydroxyl, alkoxy carbonyl, amido, alkylamido, dialkylamido, nitro, amino, alkylamino, dialkylamino, carboxyl, thio and/or thioalkyl.

An “alkenyl” group refers, in another embodiment, to an unsaturated hydrocarbon, including straight chain, branched chain and cyclic groups having one or more double bonds. The alkenyl group may have one double bond, two double bonds, three double bonds, etc. In another embodiment, the alkenyl group has 2-12 carbons. In another embodiment, the alkenyl group has 2-6 carbons. In, another embodiment, the alkenyl group has 2-4 carbons. Examples of alkenyl groups are ethenyl, propenyl, butenyl, cyclohexenyl, etc. The alkenyl group may be unsubstituted or substituted by a halogen, hydroxy, alkoxy carbonyl, amido, alkylamido, dialkylamido, nitro, amino, alkylamino, dialkylamino, carboxyl, thio and/or thioalkyl.

An “aryl” group refers to an aromatic group having at least one carbocyclic aromatic group or heterocyclic aromatic group, which may be unsubstituted or substituted by one or more groups selected from halogen, haloalkyl, hydroxy, alkoxy carbonyl, amido, alkylamido, dialkylamido, nitro, amino, alkylamino, dialkylamino, carboxy or thio or thioalkyl. Nonlimiting examples of aryl rings are phenyl, naphthyl, pyranyl, pyrrolyl, pyrazinyl, pyrimidinyl, pyrazolyl, pyridinyl, furanyl, thiophenyl, thiazolyl, imidazolyl, isoxazolyl, and the like. In one embodiment, the aryl group is a 4-8 membered ring. In another embodiment, the aryl group is a 4-12 membered ring(s). In another embodiment, the aryl group is is a 6 membered ring. In another embodiment, the aryl group is a 5 membered ring. In another embodiment, the aryl group is 2-4 fused ring system.

A “aldehyde” group refers, in one embodiment to an alkyl, or alkenyl substituted by a formyl group, wherein the alkyl or alkenyl are as defined hereinabove. In another embodiment, the aldehyde group is an aryl, or phenyl group substituted by a formyl group, wherein the aryl is as defined hereinabove. Examples of aldehydes are: formyl, acetal, propanal, butanal, pentanal, benzaldehyde. In another embodiment, the aldehyde group is a formyl group.

A “haloalkyl” group refers, in another embodiment, to an alkyl group as defined above, which is substituted by one or more halogen atoms, e.g. by F, Cl, Br or I.

In one embodiment, the nanoprobe which is used in the methods provided herein, have a luminescent moiety that is a metallophthalocyanines represented by formula IV:

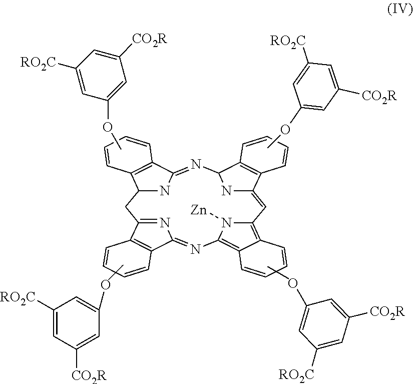

In one embodiment, the nanoprobe which is used in the methods provided herein, has a luminescent moiety that is a metallophthalocyanines represented by formula V:

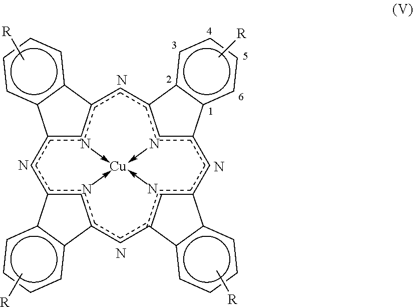

In another embodiment, the nanoprobe which is used in the methods provided herein, comprise a dendrimer or a hyperbranched polymer. In another embodiment the dendrimer possess a hydrophobic interiors and hydrophilic periphery. In another embodiment, dendrimers are internally cross-linked and, therefore, locked in fully closed conformations, completely eliminating access of quenchers within the collisional range from chromophores!—this is a very important point. I wonder is if we should somehow accentuate it.

In one embodiment, the cross-linking of the dendrimers or hyperbranched polymers used in the nanoprobes described herein, is via covalent bonds, or non-covalent bonds or their combination in other embodiments. In one embodiment, the cross-linked hyperbranched polymer or dendrimer reduces the access of a small quenching molecules to the luminescent moiety.

In one embodiment, provided herein is an imaging nanoprobe, comprising a luminescent metalloporphyrin moiety, covalently bound to a dendrimer with hydrophilic peripheral layer, whereby the dendrimer is covalently cross linked to reduce the access of water or oxygen to the luminescent moiety, thereby increasing signal to noise ratio of the nanoprobe.

In one embodiment, the term “hyperbranched polymer”, is not intended to encompass dendrimers. Dendrimers of a given generation are monodispersed in one embodiment, having a polydispersity of less than about 1.02, with highly defined globular molecules, having a degree of branching that is 100% in one embodiment, or very nearly 100% in another embodiment. In another embodiment, the term “hyperbranched polymer” refers to polymers having branches upon branches. However, in contrast to dendrimers, hyperbranched polymers may be prepared in a one-step, one-pot procedure. This facilitates the synthesis of large quantities of materials, at high yields, and at a relatively low cost. In another embodiment, the properties of hyperbranched polymers are different from those of corresponding dendrimers due; in certain embodiments, to imperfect branching and rather large polydispersities, both of which are governed mainly by the statistical nature of the chemical reactions involved in their synthesis. Therefore, in one embodiment, hyperbranched polymers are an intermediate between traditional branched polymers and dendrimers. In one embodiment, a hyperbranched polymer molecule contains a mixture of linear and branched repeating units, whereas an ideal dendrimer contains only branched repeating units. The degree of branching, which reflects the fraction of branching sites relative to a perfectly branching system (i.e., an ideal dendrimer), for a hyperbranched polymer is greater than 0 and less than 1, with values being in certain embodiments, from about 0.25 to about 0.45. Unlike ideal dendrimers which have a polydispersity of 1, hyperbranched polymers have typical polydispersities being greater than 1.1 even at a relatively low molecular weight such as 1,000 Daltons, and greater than 1.5 at molecular weights of about 10,000 or higher. These differences between the polydispersities and degree of branching of hyperbranched polymers and dendrimers are indicative of the relatively higher non-ideality, randomness, and irregularity of hyperbranched polymers as compared with dendrimers, and distinguish hyperbranched polymers from dendrimers. In one embodiment, lifetime of the luminescent moiety which is within a hyperbranched used in the nanoprobes described herein, is affected by the higher non-ideality, randomness, and irregularity of hyperbranched polymers as compared with dendrimers.

In one embodiment, the dendrons, linked to polyfunctionalized metalloporphyrins, form a well-defined surrounding environments, acting in another embodiment as shields from oxygen and other quenchers. In another embodiment, dendrimers reach the required size while maintaining nearly ideal monodispersity. Under certain circumstances, two identical phosphorescent cores surrounded by different polymeric jackets produce different analytical signals (e.g. lifetimes), causing measurement uncertainty. Therefore, monodispersity is one of the key parameters for molecular sensors, and the dendrimers described herein, are monodisperse polymers.

In another embodiment, the metalloporphirines used in the luminescent moieties described herein are extended further using π-extension, thereby shifting their peak excitation wavelength higher. Accordingly, in one embodiment, the peak wavelength of a metalloporphirine used in the nanoprobes, methods and systems described herein is shifted from about 410 nm to between about 600 to 700 nm. In another embodiment, the peak wavelength of the metalloporphirine is shifted from between about 600 and 700 nm to between about 800 to 950 nm.

In one embodiment, term “monodisperse” refers to a population of particles (e.g., a dendritic to system) wherein the particles have substantially identical size and shape. For the purpose of the present invention, a “monodisperse” population of particles means that at least about 67% of the particles, preferably about 75% to about 97% of the particles, fall within a specified particle size range. A population of monodisperse particles deviates less than 10% rms (root-mean-square) in diameter from the mean population diameter and preferably less than 5% rms.

In another embodiment, the nanoprobe which is used in the methods provided herein, comprises a peripheral polymeric layer. The peripheral layer refers in one embodiment to an outer layer which is linked to the dendrimer or to the hyperbranched polymer described herein, used in the nanoprobes described herein. In another embodiment, the peripheral layer or unit increases the solubility of the nanoprobe. In another embodiment, the peripheral layer is poly(ethyleneglycol) (PEG), poly(lactic-co-glycolic acid) PLGA, Poly-L-lactic acid (PLLA) or polysorbate, polyvinylalcohol (PVOH), or their combination in other discrete embodiments of the functionalized outer layer. In another embodiment, the functionalized outer layer is linked to an agent capable of binding to a pre-determined target, whereby in another embodiment, the agent is an antibody, or a ligand specific to an antibody, a receptor, a signaling molecule specific to a receptor, or their combination in other certain discrete embodiments of the nanoprobes described herein.

In one embodiment, this invention provides a nanoprobe and methods of use thereof. In another embodiment, the nanoprobe is represented by the general formula A:

C-(AGⁿ-R)ₘ

(A)

where:
C is a luminescent moeity core;
AG is the dendritic aryl-glycine skeleton;
n is the generation number;
R is peripheral unit; and
m is the number of dendritic wedges attached to the core

In one embodiment, C of the nanoprobe A, comprise a luminescent moeity. In another embodiment, the luminescent moeity is a metalloporphyirin of formula I. In another embodiment, the metal of said metalloporphyrine is palladium or platinum ions. In another embodiment, C is a luminescent moeity of formula II. In another embodiment, C is a luminescent moeity of formula III. In another embodiment, C is a luminescent moeity of formula IV. In another embodiment, C is a luminescent moeity of formula V.

In another embodiment, the nanoprobes provided herein, used in the methods described, have the

C^{π}-(D_{c}ⁿ-R)ₘ,

where C is a dendritic core, p is the number of p-extensions of the porphyrine core, D_{c} is a cross-linked dendritic skeleton, n is a generation number, R is the polymeric unit which optionally is linked to a moiety specific to the pre-determined target and m is the number of dendritic wedges attached to the core. In one embodiment, AG of the nanoprobe A, is a dendritic aryl-glycine skeleton.

In one embodiment, n of nanoprobe A, is the generation number of the dendrimeric structure. In another embodiment n is between 1 and 5. In another embodiment, n is 1. In another embodiment, n is 2 In another embodiment, n is 3. In another embodiment, n is 4. In another embodiment, n is 5.

In one embodiment, R of the nanoprobe A, is a peripheral polymeric unit. In one embodiment, R forms a polymer layer which embeds the dendritic structure and its core. In another embodiment, the peripheral layer is poly(ethyleneglycol) (PEG), poly(lactide-co-glycolide) acid (PLGA), poly(L-lactide) acid (PLLA), poly(D-lactide) acid (PDLA), polyvinylalcohol (PVOH) or polysorbate.

In one embodiment, m of the nanoprobe A is the number of dendritic wedges. In one embodiment m is between 4 and 10. In another embodiment m is between 6 and 8. In another embodiment m is 6. In another embodiment m is 7. In another embodiment m is 8. In another embodiment m is 9. In another embodiment m is 10. In one embodiment, C, the core is a metalloporphyrine molecule, which in another embodiment has undergone π-extension. In one embodiment, the nanoprobe having a palladium metalloporphyrine core, has undergone π-extension (π=1), or no π-extension (π=0), 2 π-extensions (π=2), or 3 π-extensions (π=3) in other discrete embodiments of the number of π-extensions of the metalloporphyrine core used in the nanoprobes described herein and used in the methods provided.

In one embodiment, the nanoprobes of this invention possess the following properties:
Absorption and emission in the NIR region of the spectrum;
High extinction coefficients (up to 250,000 cm-1M⁻¹) in the NIR region;
High phosphorescence quantum yields (up to 50%);
Long emission lifetimes (up to 1 ms (10⁻³ s);
Large Stokes shifts (˜4,000 cm⁻¹);
Small molecular size: probes in solution adopt globular or elliptical conformations with diameters of less than 5 nm.

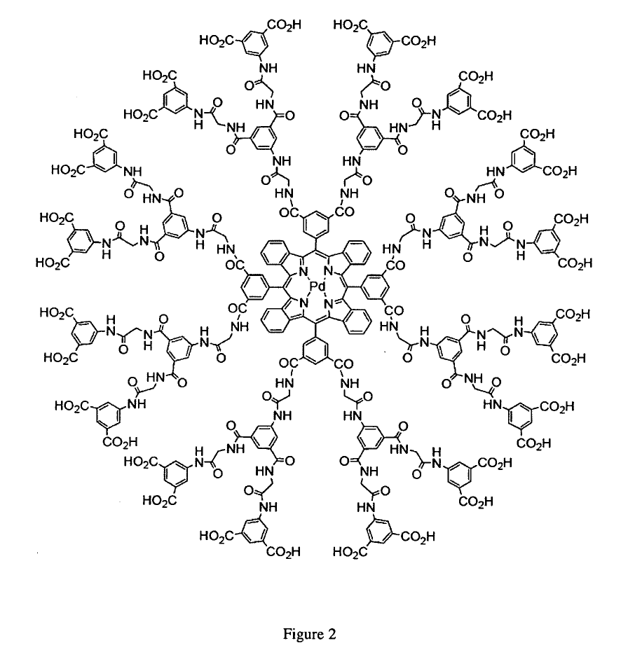

In one embodiment a metalloporphirin embedded in a dendrimer is shown in FIG. 2. FIG. 2 shows a Pd-tetraphenyltetrabenzoporphyrin (PdTBP) and eight G2 (generation two) arylglycine (AG) dendrons, whose carboxyl groups form the peripheral layer.

In one embodiment, a process for the preparation of a core metalloporphyrin embedded in a dendron comprises coupling reaction between the metalloporphyrin and the end the dendron. For example, a butoxy terminated AG³-dendron with focal amino group is abbreviated as H₂N-AG³-OBu. Coupling of eight H₂N-AG³-OBu wedges to the core porphyrin (e.g. PtP) is accomplished using peptide coupling protocol as described herein below:

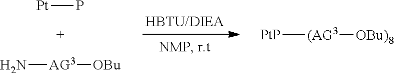

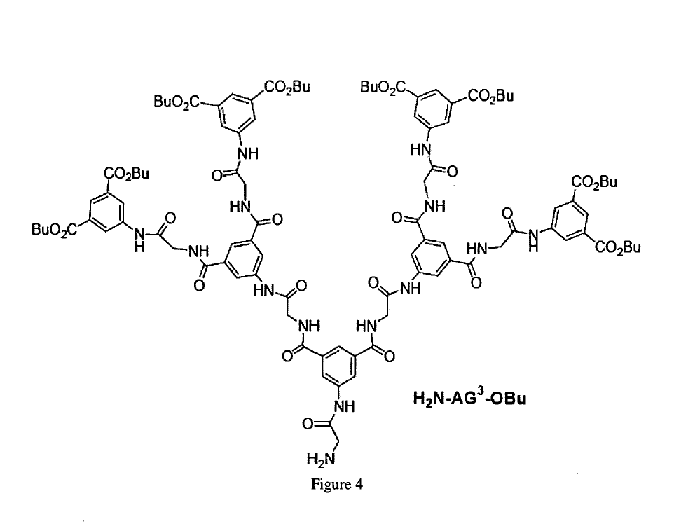

An example of G3 AG-dendron is shown in FIG. 4. In one embodiment, synthesis of AG-dendrons is done, based on the classic Fischer haloacylhalide method. This method bypasses all expensive peptide-coupling reagents and employs only trivial bulk chemicals, such as aminoisophthalic acid, chloroacetylchloride, SOCl₂, soda and ammonia.

In one embodiment, a process for the preparation of the dendron attached to the core comprise preparation of dendrons terminated by ester groups, e.g. butyl esters, which provide optimal solubility for synthetic and purification purposes. Later, these groups are hydrolyzed and either left as free carboxyls in one embodiment, or in another embodiment, converted to neutral hydrophilic groups (e.g. polyethyleneglycol esters) or modified into more specific groups in yet another embodiment, thereby permitting coupling to the site of interest. In one embodiment, carboxyl groups on the dendrimer periphery are reacted with ethylenediamine (EDA), resulting in a dendrimer with multiple amino-groups (the following scheme provides modification of the dendrimer periphery):

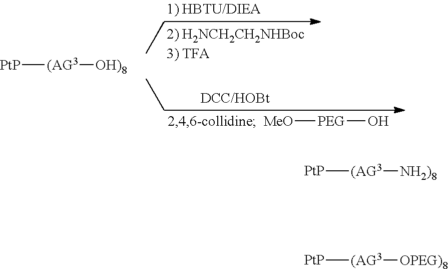

HBTU-2-(1H-benzotriazole-1-yl)-1,1,3,3-tetramethyluronium hexafluorophosphate; DIEA-N,N-Diisopropylethylamine; TFA-Trifluoroacetic acid; DCC—N,N′-dicyclohexylcarbodiimide; HOBt-1-hydroxybenzotriazole; MeO-PEG-OH-polyethylene glycol monomethylether (Av. M_{w} 350 Da).

The hyperbranched polymers as described herein are prepared in one embodiment, by any applicable polymerization method, including but not limited to: (a) monomolecular polymerization of AₓB_{y}C_{z} monomers, wherein A and B are moieties that are reactive with each other but not significantly reactive with themselves, x and y are integers having a value of at least 1 and at least one of x or y has a value of at least 2, C is a functional group that is not significantly reactive with either the A or B moieties or itself during polymerization of the hyperbranched polymer and z is an integer having a value of 1, or greater; (b) copolymerization or bi-molecular polymerization of AₓB_{y}C_{z} and B_{y} monomers, wherein A, B and C are moieties as defined above, x and y are integers one of which having a value of at least 2 and the other having a value greater than 2, and z is an integer having a value of at least 1; and (c) multi-molecular polymerization reactions of two or more polyfunctional monomers, wherein the is functionality of A or B is at least 2, while the functionality of at least one of A or B is higher than 2 (e.g., A₂+A₂C_{z}+B₃). Also provided herein are other synthetic strategies wherein one or more monomers used in the synthesis of hyperbranched polymers contains a latent functional group or groups that do not react significantly under the polymerization conditions. For example, two different monomers each having a latent functional group of the same or different type can be reacted to form a hyperbranched polymer in accordance with this invention (i.e., AₓB_{y}C_{z}+B_{y}C_{w} or AₓB_{y}C_{z}+B_{y}D_{w}, wherein A, B, C, x, y, and z are as defined above, and D is a second kind of latent functional group that does not react significantly during the A+B polymerization and w is an integer having a value of at least 1). Also, a single monomer (e.g., AₓB_{y}C_{z}D_{w}) having x number of A groups and y number of B groups that react with each other during the polymerization and z number of C groups and w number or D groups can be polymerized to form a hyperbranched polymer containing two different types of latent functional groups that are not reactive during the A+B polymerization but are reactive under another set of conditions. In each of the above examples, at least one of x and y must be an integer equal or greater than 2 in order to form a hyperbranched polymer. Other synthetic strategies that may be employed may include any of the preceding systems involving more than two types of reacting functional groups and/or systems involving simultaneous polymerization reactions, such as multi-bond opening or ring opening reactions, step-growth polycondensations or polyadditions, and chain-growth polymerizations.

In one embodiment, peripheral groups on the dendrimer account for interactions of the probe with the measurement environment and, in another embodiment, permit coupling of the probe to the analyte of interest to a pre-determined target. In another embodiment, the agent is an antibody, or a ligand specific to an antibody, a receptor, a signaling molecule specific to a receptor, or their combination in other certain discrete embodiments of the nanoprobes described herein.

The imaging target to of the nanoprobes provided herein, is a cell in one embodiment, or a virus or any biological macromolecule in other discrete embodiments of the targets for which the nanoprobes described herein are used.

In one embodiment, the nanoprobes described herein are used in the optical imaging systems described herein. In another embodiment, provided herein is an optical imaging system comprising: an electronic imaging device configured to capture an image of a predetermined site; a nanoprobe comprising a lumisescent moeity with a long excited state lifetime, embedded in a dendrimer with a peripheral; and a projector configured to project a visible representation of the captured image.

In another embodiment, the optical imaging system uses a computer to process the emitted data and its display, as well as store the data in a memory module thus allowing for a later query of the obtained images. In one embodiment, the predetermined site sought to be imaged is a tumor, or a lesion, a digestive tract, a lymph node, a brain tissue, a lung tissue or a nervous system tissue in other discrete embodiments of the sites sought to be imaged using the optical imaging systems described herein.

In one embodiment, the optical imaging systems described herein, further comprise an excitation light source capable of providing one or more wavelengths to excite the nanoprobe used therein. In one embodiment, the excitation source used in the methods and optical imaging systems described herein is capable of pulsing light at varying wavelength corresponding to the peak excitation wavelength of the nanoprobes used. In one embodiment, the excitation source used in the methods and optical imaging systems described herein is capable of pulsing light over a period that is shorter than or equals to the luminescence lifetime of the luminescent moiety at zero quencher concentration. In another embodiment, measuring the emitted light in the methods and optical imaging systems described herein is performed after a delay following the excitation pulse, but prior to the end of the luminescence decay, thus eliminating the background signal and increasing the signal-to-noise ratio (SNR).

In another embodiment, the electronic imaging device used in the optical imaging systems, that are used in another embodiment to perform the methods described herein comprises a computer assisted tomography (CAT), magnetic resonance spectroscopy (MRS), magnetic resonance imaging (MRI), positron emission tomography (PET), single-photon emission computed tomography (SPECT), bioluminescence image (BLI) or their equivalent.

In one embodiment, the excitation source used in the methods and optical imaging systems described herein further comprising a filter that selectively transmits a predetermined wavelength. In one embodiment, the excitation source used in the methods and optical imaging systems described herein is capable of providing a discrete light wavelength in the range of between about 410 and 960 nm. In another embodiment the excitation source used in the methods and optical imaging systems described herein is capable of providing a discrete light wavelength in the range of between about 410 and 960 nm, or between 190 and 2400 nm in another embodiment, or between 200 and 400 nm in another embodiment, or between 400 and 500 nm in another embodiment, or between 500 and 600 nm in another embodiment, or between 600 and 700 nm in another embodiment, or between 700 and 800 nm in another embodiment, or between 800 and 900 nm in another embodiment, or between 900 and 960 nm in another embodiment, or between 900 and 1000 nm in another embodiment, or between 1000 and 1400 nm in another embodiment, or between 1400 and 1800 nm in another embodiment, or between 1800 and 2400 nm in another embodiment, or their combination, each a discrete embodiment of the excitation wavelength emitted by the excitation light source used in the methods and systems described herein.

In one embodiment, the methods provided herein make use of more than a single nanoprobe type. Accordingly, in one embodiment provided herein is a method of imaging a pre-determined target in a subject, comprising: administering to the subject a nanoprobe mixture comprising: a lumisescent moeity with a long excited state lifetime, embedded in a dendrimer within a polymeric layer, wherein said dendrimer is thermodynamically incompatible with a measurement environment, having a quenching coefficient of between about 50 and 180 mm Hg⁻¹ s⁻¹ and an excited state lifetime of between about 50 and 250 μs; exciting said luminescent moeity, wherein the nanoprobes used in the mixture have different excitation and emission wavelengths and are attached peripherally to different target binding moieties; measuring the emitted light intensity from said luminescent moiety; and comparing the emitted light intensity to a standard, whereby the intensity signal and its wavelength is typical of the predetermined target.

In another embodiment, a pre-determined target is a cell, mitochondria, lysozymes, virus, a tumor or a biological macromolecule.

In one embodiment, provided herein is a pharmaceutical formulation comprising the nanoprobe wherein the nanoprobe comprises lumisescent moeity embedded in a dendrimer or a hyperbranched polymer, wherein said dendrimer or hyperbranched polymer is embedded within a polymeric layer; wherein the formulation is suitable for administration as an imaging agent (e.g., for intravenous injection).

In one embodiment, this invention provides a method for in vivo imaging a tumor neovasculature in an individual comprising (i) administering a nanoprobe a lumisescent moeity with a long excited state lifetime, embedded in a dendrimer, wherein said dendrimer is embedded within a peripheral layer; (ii) exciting said luminescent moeity by directing light; and (iii) detecting light emitted from said tumor neovasculature.

Neovascularization is essential for tumor growth and metastasis. Angiogenic phenotype is an early event in tumorigenesis, allowing tumors to grow beyond the size otherwise limited by the diffusion of oxygen and other nutrients through tissue. The identification of families of endogenous pro- and anti-angiogenic factors can provide therapeutic approaches aimed at interrupting tumor blood supply.

In another embodiment, the methods of this invention provides administering the nanoprobe of this invention. In another embodiment, the nanoprobe is administered by intravenous injection. In another embodiment, the nanoprobe is administered orally as a liquid suspension or as a liquid solution.

In one embodiment, the methods of this invention comprise an excitation step. In another embodiment, the excitation step is in a form of a light pulse, which is shorter than or equals to the luminescence lifetime of the luminescent moiety.

In another embodiment, the methods of this invention provides a detecting step. In another embodiment, the detecting step is performed after a delay following the excitation pulse, but prior to the end of the luminescence decay, thus eliminating the background signal and increasing the signal-to-noise ratio. In another embodiment the luminescence is detected by high resolution microscopic imaging. In another embodiment, the luminescence is detected by near infrared 3D tomography. In another embodiment, the luminescence is phosphorescence

In one embodiment, the nanoprobes described herein, which are used in the methods provided herein, have a luminescent moiety that is a metalloporphyrin. In another embodiment, the Metalloporphyrin core of the probe (FIG. 2) accounts for its spectral properties. Molar extinction coefficients (ε) of porphyrins place them among the highest known organic molecules, e.g. reach up to 250,000 M⁻¹ cm⁻¹ in the Vis-NIR range. Phosphorescence quantum yields (φₚ) of Pt and Pd porphyrins in deoxygenated solutions at room temperature are in the range of 0.1-0.2, but reach as high as 0.5 for some π-extended porphyrins, e.g. Pt tetrabenzoporphyrins. As a result, in one embodiment, brightness of metalloporphyrins is extremely high. Because Pt and Pd porphyrins emit from their triplet states, the emission Stokes shifts are large, permitting in another embodiment a very efficient discrimination between emission and excitation photons. In one embodiment, the term “excitation wavelength” refers to electromagnetic energy having a shorter wavelength (higher energy) than that of the peak emission wavelength of the nanoprobes described herein.

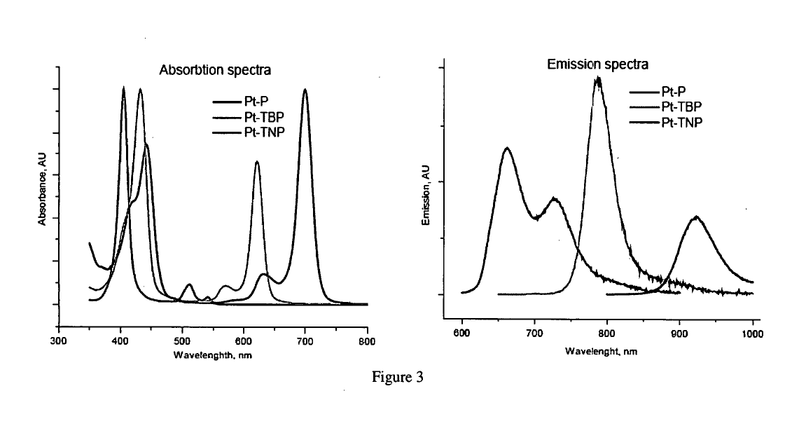

In one embodiment, the methods of this invention comprise an excitation step of said luminescence moiety of the nanoprobe of this invention. The excitation is conducted at the absorption band of the luminescence moiety. In another embodiment, the luminescence moiety is a metalloporphyrin comprising a palladium or platinum metal ions. Table I details the range of optical transitions of Pt and Pd porphyrins. Absorption bands of porphyrins (FIG. 3) cover practically the entire optical spectrum: from near UV, through visible to NW region. In one embodiment, any one of the porphyrins shown in Table I is used in the nanoprobes described herein.

TABLE I
Phosphorescent metalloporphyrins used as cores for dendritic luminescent labels

| | Octacarboxyporphyrin | |
|---|---|---|
| | 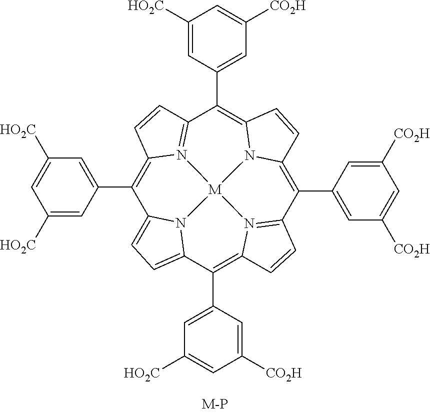 | |
| Porphyrin M | Pt | |
| Absorption λₘₐₓ, nm (log e) | 406 (5.54) 512 (4.50) | |
| Phosphorescence λₘₐₓ, nm | 664, 725 | |
| τ₀, μs | 53 | |
| φₚ | 0.24 | |
| | Octacarboxytetra benzoporphyrin | |
| | 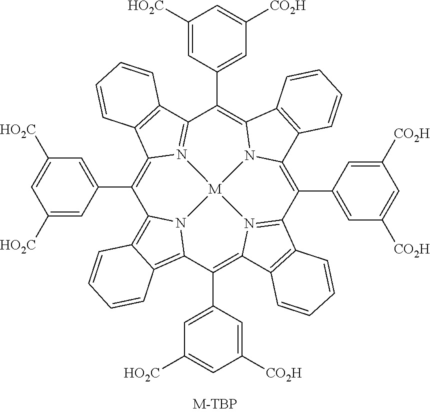 | |
| Porphyrin M | Pt | Pd |
| Absorption λₘₐₓ, nm (log e) | 433 (5.23) 622 (5.00) | 446 (5.46) 635 (5.00) |
| Phosphorescence λₘₐₓ, nm | 791 | 816 |
| τ₀, μs | 41 | 210 |
| φₚ | 0.30 | 0.09 |

Octacarboxytetra naphthoporphyrin

| | 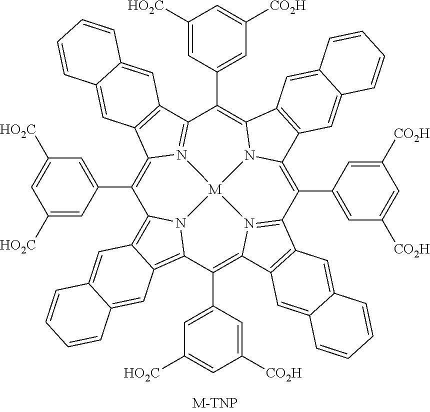 |
|---|---|
| Porphyrin M | Pt |
| Absorption λₘₐₓ, nm (log e) | 441 (5.14) 700 (5.28) |
| Phosphorescence λₘₐₓ, nm | 923 |
| τ₀, μs | 12 |
| φₚ | 0.06 |

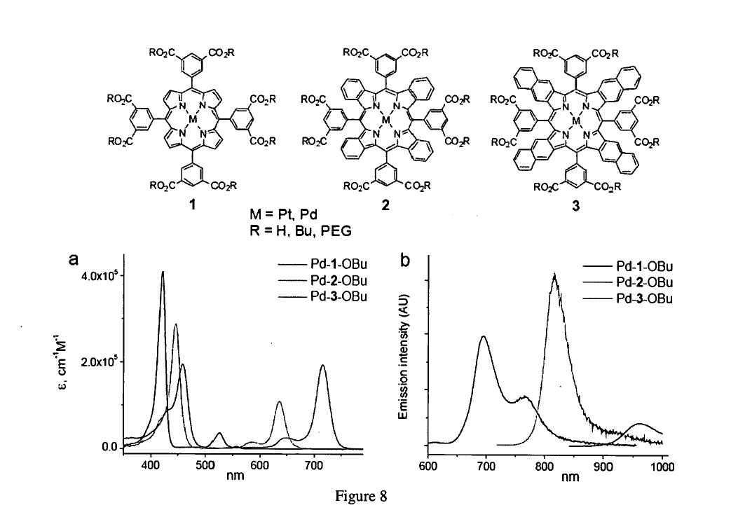

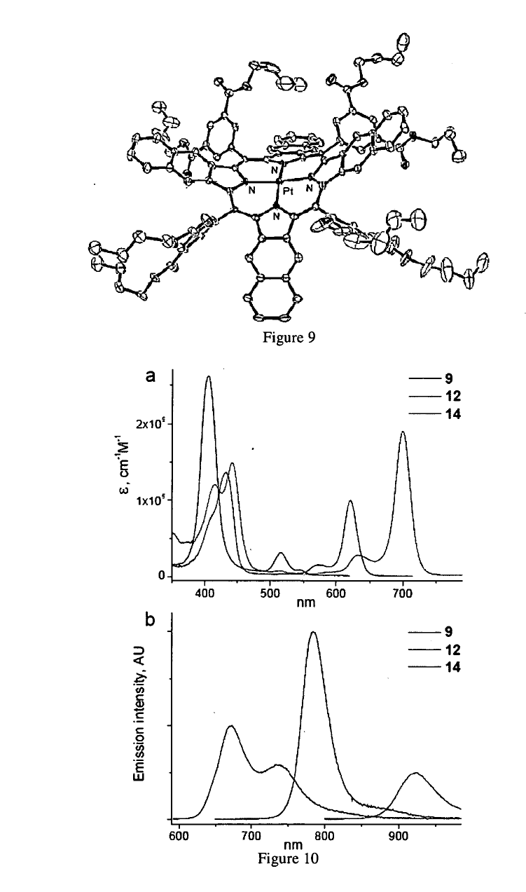

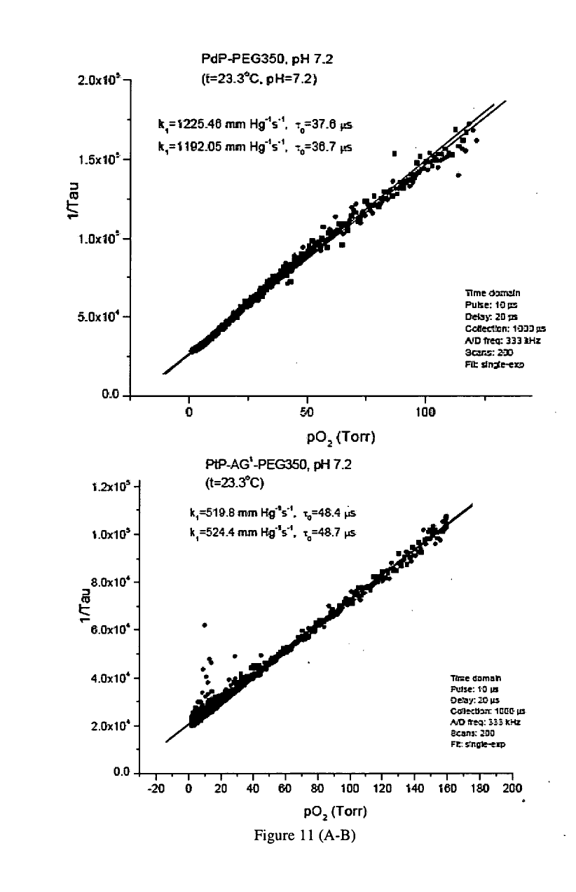

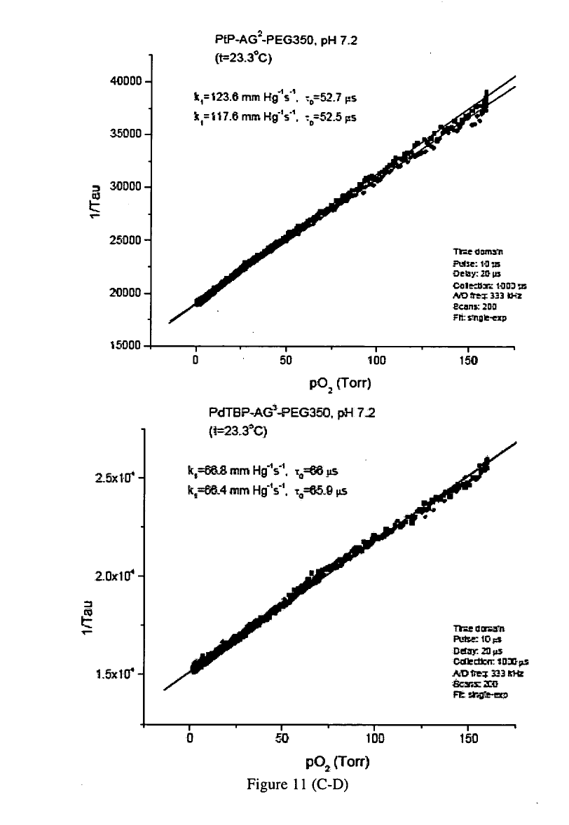

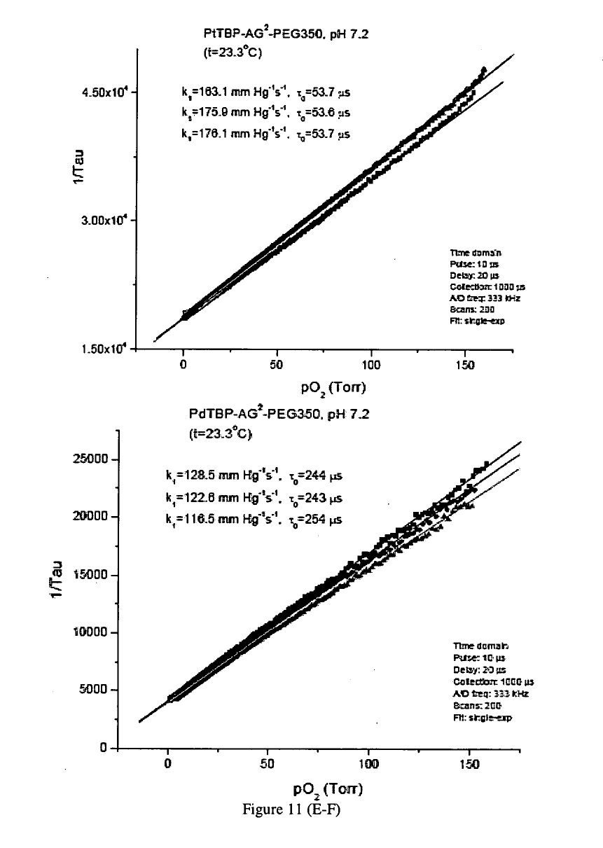

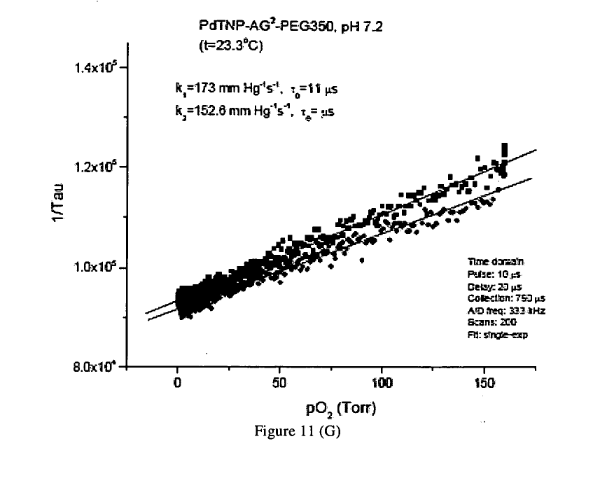

In Pt and Pd porphyrins, S1→T1 intersystem crossing is in one embodiment the deactivation pathway of the singlet excited states (S1), and the resulting triplet states phosphorescent. In another embodiment, Pt and Pd meso-tetraarylaryl tetrabenzoporphyrins (TBP's) used in the nanoprobes, sensors and methods described herein, although highly non-planar, phosphoresce with high quantum yields. Pd and Pt tetraaryltetranaphthoporphyrins (FIG. 8 and Example 1) are also non-planar and in addition have much narrower T1-S0 gaps (Table II). In another embodiment the absorption bands of Pt and Pd P's, TBP's and TNP's cover the entire UV-vis-NIR range, providing in one embodiment multiple excitation wavelengths. In one embodiment, the absorption Qbands of TBP's and TNP's used in the sensors, nanoprobes and methods described herein are shifted to the red, i.e. into the region between ˜630 and ˜950 nm, where the absorption of endogenous chromophores is significantly lower, thereby providing nanoprobes capable of distinguishing endogenous chromophores from the chromophores provided with the nanoprobes and sensors described herein. The oscillator strengths of these bands are extremely high, making TBP's and TNP's especially well suitable in another embodiment for optical tomographic applications. Accordingly and in one embodiment, provided herein is a tomographic imaging nanoprobe, comprising a Pd-TNP moiety having an excitation wavelength between about 630 and 950 nm, operably linked within the interior of a dendrimer or a hyperbranched polymer; within a functionalized outer layer, In another embodiment the luminescent moiety is PtP, or in another embodiment PdP, or in another embodiment PtTBP, or in another embodiment PdTBP, or in another embodiment PtTNP, each a discrete embodiment of the luminescent moiety used in the tomographic imaging nanoprobes and sensors provided herein.

In another embodiment, the excited state lifetime of the luminescent moiety at zero Oxygen concentration, operably linked within the interior of a dendrimer or a hyperbranched polymer used in the nanoprobes, sensors and methods described herein is a luminescent moiety with long excited state lifetime of between about 50 and 250 μs. In another embodiment, the excited state lifetime of the luminescent moiety at zero Oxygen concentration is between about 50-100 μs, or between about 100-150 μs, or between about 150-175 μs, or between about 175-200 μs, or between about 200-225 μs, or between about 225-250 μs, each a discrete embodiment of the excited state lifetime of the luminescent moiety at zero Oxygen concentration, operably linked within the interior of a dendrimer or a hyperbranched polymer used in the nanoprobes, sensors and methods described herein.

In one embodiment the folding of the dendrimer branches or the hyperbranched polymer in another embodiment creates a microenvironment where the free volume, referring in one embodiment to the macromolecular volume not occupied by the molecules or their interconnecting bonds, is smaller than the volume of the critical segment of the dendrimer branches or that of the hyperbranched polymer. Effectively, this environment restricts translational mobility in one embodiment, or rotational mobility in another embodiment, thereby restricting the quenching molecule's ability to diffuse through the dendritic core to the luminescent moiety, by either repetition or through jumping through voids. In another embodiment, reduction in free volume is affected by cross-linking the hyperbranched polymer or in another embodiment, by cross-linking the dendritic wedges and effectively increases the activation energy necessary for the diffusing quencher to separate the polymer chains, thus creating the voids necessary for its collision with the luminescent moiety. In one embodiment, the net effect of the cross-linking or decrease in free volume is that the excited state lifetime is longer than the diffusion time of the quenching molecule to the chromophore embedded in the dendrimer, making the probes described herein, insensitive to the presence of the quencher molecule, such as Oxygen in one embodiment.

In one embodiment, the term “Critical segment” refers to the minimum number of molecules capable of translational or rotational mobility. In another embodiment, the critical segment refers to the Kuhn statistical segment. In another embodiment, the conformation of a polymer chain in solution resulting, among others, from polymer-solvent interactions depends strongly on the dynamics of the length scale of the critical segment size. Polymer chains are modeled in one embodiment as a freely-jointed chain of N segments, each of length l, where each segment may contain several monomers. In one embodiment, the term “Translational mobility” refers to the ability of the critical segment to translate in location. In another embodiment, the term “Rotational Mobility” refers to the ability of the critical segments to rotate freely about the bonds in the joint linking one segment to another. The threshold temperature marking the onset of rotational mobility is referred in one embodiment is the β-relaxation temperature. In another embodiment, the onset of translational mobility is referred to as the glass transition temperature (Tg), or in another embodiment, as the α-relaxation onset temperature. In one embodiment, using the arylglycine (AG) polymer branches in a polar environment, forces the dendrimers into a supercooled melt liquid phase where mobility is restricted to the point where at physiological temperature the polymer is below its corresponding α-relaxation onset, or in another embodiment, below its β-relaxation onset temperature. In another embodiment, crosslinking the to dendrimer core, or the hyperbranched polymer core, in the nanoprobes, sensors and methods described herein reduces the size of the critical segment length to the point where it is about the size of the diffusing quenching molecule.

In one embodiment, the phosphorescence lifetimes (τ₀) and the quantum yields (φ) of the nanoprobes and sensors, which in another embodiment are used in the methods described herein increase with an increase in the dendrimer generation. In another embodiment the quantum yield grow consistently from G0 to G3 dendrimers.

In one embodiment, quenching of the probe's excited state requires direct collision of the quencher with the chromophore. Likewise, it is the diffusion of the quencher to the chromophore that determines the total quenching rate. A major factor determining the rate of diffusion of small molecules is the media dynamics wherein diffusion takes place. Restricting the medium dynamics in the vicinity of the chromophore using the nanoprobes described herein, slows down the diffusion and reduce quenching. In another embodiment, the use of the dendritic jackets described herein, in the nanoprobes described herein, to control the diffusion of small molecules in the chromophore's vicinity, i.e. to construct impermeable molecular shells around phosphorescent chromophores.

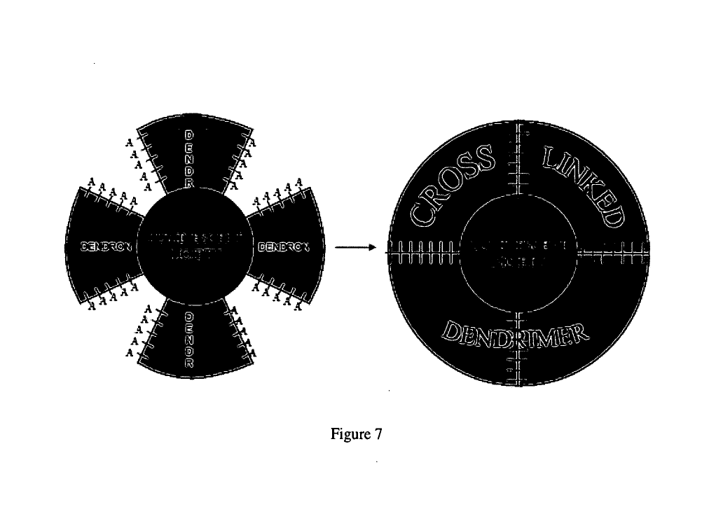

In another embodiment, mobility of the medium comprising the immediate vicinity of the chromophore is further restricted by cross-linking the dendritic matrix. Cross-linking can originate in certain embodiments of the nanoprobes provided herein, from any covalent or non-covalent interactions within the dendrimer structure. In one embodiment, AG-dendrimers (FIG. 4) are tightly cross-linked by introduction of reactive groups into the dendron skeleton and reacting these groups after the dendrimer assembly. Such process will construct in another embodiment, a network of covalent bonds within the dendrimer, significantly restricting the dendrimer dynamics (See e.g. FIG. 7).

In one embodiment, the nanoprobes comprising a luminescent moiety, operably linked within the interior of a dendrimer or a hyperbranched polymer, within a functionalized outer layer described hereinabove, are used in the methods described herein. Accordingly and in another embodiment, provided herein is a method of imaging a pre-determined target in a subject, comprising the step of contacting the subject with the nanoprobe comprising a luminescent moiety, operably linked within the interior of a dendrimer or a hyperbranched polymer, which is within a functionalized outer layer, whereby the functionalized outer layer is within an agent capable of binding to a pre-determined target; exposing the luminescent moiety to an electromagnetic radiation; and detecting the luminescence of the luminescent moiety.

In one embodiment, the electromagnetic radiation used in the step of exposing the luminescent moiety in the methods described herein, is delivered in the form of a light pulse, which is shorter than or equals to the luminescence lifetime of the luminescent moiety.

In one embodiment, the term “luminescence” refers to the process of emitting electromagnetic radiation (e.g., light) from an object. Luminescence results when a system undergoes a transition from an excited state to a lower energy state, with a corresponding release of energy in the form of a photon. These energy states can be electronic, vibrational, rotational, or any combination thereof. The transition responsible for luminescence is stimulated in one embodiment, through the release of energy stored in the system chemically, kinetically, or added to the system from an external source. The external source of energy can be of a variety of types including chemical, thermal, electrical, magnetic, electromagnetic, or physical, or any other type of energy source capable of causing a system to be excited into a state higher in energy than the ground state. In one embodiment, a system is excited by absorbing a photon of light produced by a light pulse, by being placed in an electrical field, or through a chemical oxidation-reduction reaction. The energy of the photons emitted during luminescence can be in a range from low-energy microwave radiation to high-energy X-ray radiation. In another embodiment, luminescence refers to electromagnetic radiation in the range from UV to IR radiation, and in one embodiment, refers to visible electromagnetic radiation (i.e., light).

The term “about” as used herein means in quantitative terms plus or minus 5%, or in another embodiment plus or minus 10%, or in another embodiment plus or minus 15%, or in another embodiment plus or minus 20%.

The term “subject” refers in one embodiment to a mammal including a human in need of therapy for, or susceptible to, a condition or its sequalae. The subject may include dogs, cats, pigs, cows, sheep, goats, horses, rats, and mice and humans. The term “subject” does not exclude an individual that is normal in all respects.

The following examples are presented in order to more fully illustrate the preferred embodiments of the invention. They should in no way be construed, however, as limiting the broad scope of the invention.

### EXAMPLES

### Example 1

### Dendritically Protected Phosphorescent Probes with Single Coupling Site

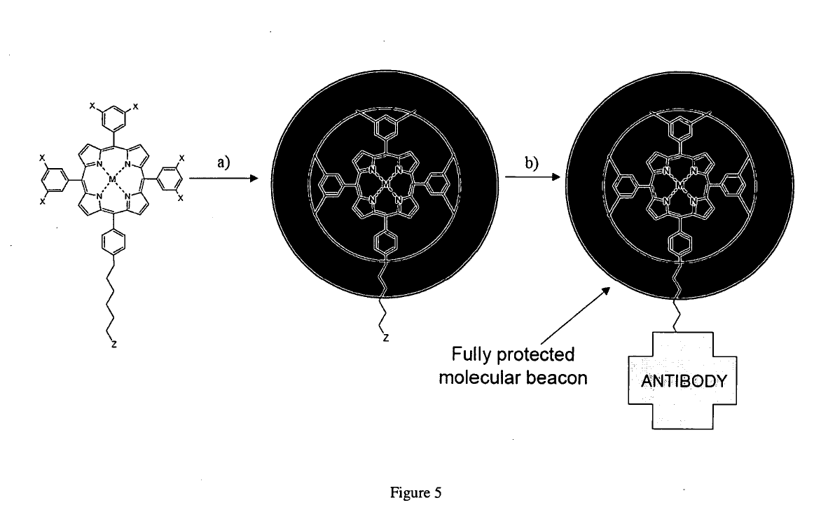

A method of building protected phosphorescent probes with single attachment sites (Z) is shown in FIG. 5. Using these strategies, one anchor site on the porphyrin is kept protected (group Z), while others (groups X) are available for dendrimerization. Deprotection of the protected site after dendrimerization leads to a phosphorescent label with a single attachment site. Such constructs are used for selective bio-labeling.

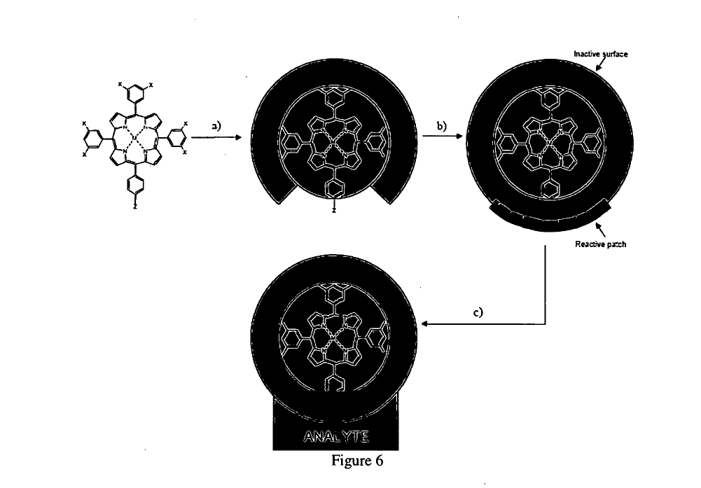

Another variant of this approach includes modification of one out of eight anchor groups on the metalloporphyrin with a dendron having other peripheral groups than dendrons attached to the remaining seven anchor points. Such a scheme is accomplished by using orthogonal derivatization chemistries. The resulting dendrimer possess not one, but several functional groups, suitable for reaction with e.g. antibodies, but all these groups are localized in one section of the dendrimer boundary, resembling a VELCRO™ label on the surface of a tennis ball. An illustration of this design is shown in FIG. 6.

The described above strategies makes it possible to selectively label analytes of interest in a selective and specific fashion.

### Example 2

### “Protected” Luminescent Probes

All solvents and reagents were obtained from commercial sources and used as received. Pd porphyrins Pd-1-OBu and Pd-1-OH were synthesized as described previously. Column chromatography was performed on Selecto™ silica gel (Fisher) or aluminum oxide (neutral, Brockmann I, ˜150 mesh, 58 Å). Preparative GPC was performed on S—X1 (Biorad) beads, using THF as a mobile phase, unless otherwise stated. ₁H and ₁₃C NMR spectra were recorded on a Brucker DPX-400 spectrometer. Mass-spectra were obtained on a MALDI-TOF Voyager-DE™ RP BioSpectrometry workstation, using α-cyano-4-hydroxycinnamic acid as the matrix.

Quartz fluorometric cells (Starna, Inc, 1 cm optical path length) were used in optical experiments. Optical spectra were recorded on a Perkin-Elmer Lambda 35 UV-Vis spectrophotometer. Steady state fluorescence and phosphorescence measurements were performed on a SPEX Fluorolog-2 spectrofluorometer (Jobin-Yvon Horiba), equipped with an infra-red enhanced R2658P PMT (Hamamatsu). Emission spectra were obtained using solutions with absorption at the excitation maxima of approximately 0.05 OD. Quantum yields of emission of all the synthesized compounds were measured relative to the fluorescence of tetraphenylporphyrin (φ_{fl}=0:11 in deox. C₆H₆).

### Synthesis

### Synthesis of Core Porphyrins

### Regular (Non-Extended) Porphyrins:

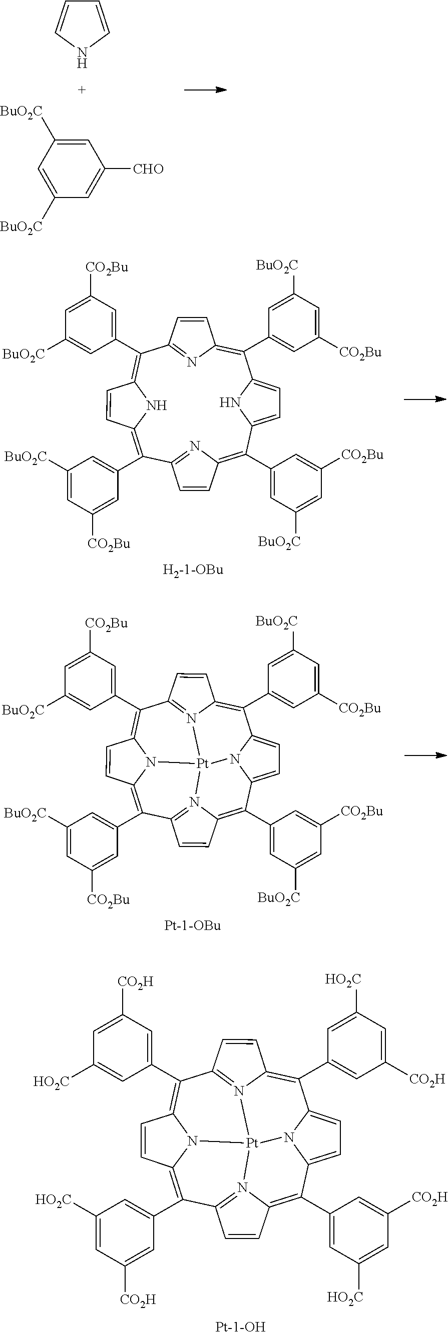

H2-1-OBu: Free base porphyrin H2-1-OBu was prepared following the general procedure described by Lindsey et al. A mixture of pyrrole (330 mg, 5 mmol) and 3,5-dibutoxycarbonylbenzaldehyde (1.53 g, 5 mmol) in CH₂Cl₂ (500 ml) was bubbled with Ar for 10 min, then BF3□Et2O (71 mg, 0.5 mmol) was added. The reaction vessel was shaded from the ambient light and left to stir for 2 h at r.t. DDQ (0.85 g, 3.75 mmol) was added, and the mixture was left overnight under stirring. The resulting solution was evaporated to dryness and suspended in MeOH (100 ml). Precipitate was filtered off and chromatographed on neutral alumina (120 g, CH₂Cl₂). The carmine-red band was collected, the solvent evaporated and the product was obtained as a red-brown powder. Yield: 576 mg, 33%.

¹H NMR (CDCl3) δ 9.16 (t, J=1.7 Hz, 4H), 9.06 (d, J=1.7 Hz, 8H), 8.80 (s, 8H), 4.46 (m, 16H), 1.80 (m, 16H), 1.48 (m, 16H), 0.96 (t, J=7.5 Hz, 24H).

¹³C NMR (CDCl3) δ 166.0, 142.5, 138.2, 131.6 (br), 131.1 (br), 129.8, 118.5, 65.6, 30.7, 19.2, 13.7.

MALDI-TOF (m/z): calculated for C₈₄H₉₆N₄O₁₆: 1415.7. Found: 1415.8, 1416.9 [M⁺; M⁺H⁺].

Pt-1-OBu: PtCl_{2□}2PhCN (707 mg, 1.5 mmol) was added to a boiling solution of H₂-1-OBu (707 mg, 0.5 mmol) in dry PhCN (300 ml), and the solution was refluxed under Ar until the conversion was complete (controlled by UV-Vis spectroscopy, typically around 5 h). To complete the conversion, an additional portion of PtCl2□2PhCN(˜200 mg) could be required. The reaction time and the required amount of PtCl_{2□}2PhCN were found to be dependent on the solvent (PhCN) purity.

The mixture was evaporated to dryness, and the residual solid was purified by column chromatography on silica gel (200 g of silica gel, CH₂Cl₂, then CH₂Cl₂/THF 20/1). An orange band was collected, the solvent was evaporated, and the residual was precipitated from CH₂Cl₂ (30 ml) by addition of MeOH (150 ml). The precipitate was separated by centrifugation and dried in vacuum. The product was obtained as an orange powder. Yield: 780 mg, 97%.

¹H NMR (CDCl₃) δ 9.13 (dt, J1=1.6 Hz, J2=0.4 Hz, 4H), 8.99 (dd, J1=1.6 Hz, J2=0.4 Hz, 8H), 8.69 (s, 8H), 4.44 (t, J=6.6 Hz, 16H), 1.78 (m, 16H), 1.46 (m, 16H), 0.94 (t, J=7.3 Hz, 24H).

¹³C NMR (CDCl3) δ 165.9, 141.7, 141.0, 137.6, 131.1, 130.5, 129.9, 120.7, 65.6, 30.7, 19.2, 13.7.

MALDI-TOF (m/z): calcd. for C₈₄H₉₂N₄O₁₆Pt: 1608.7. Found: 1608.6 [M⁺].

Pt-1-OH: Pt-1-OBu (300 mg, 0.187 mmol) was dissolved in THF (50 ml). For complete reaction it is critically important to fully dissolve the ester before addition of the reagents. KOH (˜500 mg), MeOH (5 ml) and water (0.5 ml) were added, and the mixture was stirred at r.t. until the insoluble potassium salt of the porphyrin-acid precipitated, leaving the supernatant colorless. The precipitate was decanted and dissolved in water (30 ml). The solution was acidified with conc. HCl. The orange precipitate was washed with water and dried in vacuum. The product was obtained as orange powder. Yield: 210 mg, 97%.

¹H NMR (DMSO-d6) δ 8.97 (t, J=1.5 Hz, 4H), 8.88 (d, J=1.4 Hz, 8H), 8.78 (s, 8H). 13C NMR (DMSO-d6) δ 166.9, 141.3, 140.9, 137.6, 131.6, 131.2, 130.4, 121.1.

Synthesis of Tetrabenzoporphyrins and their Pt Complexes:

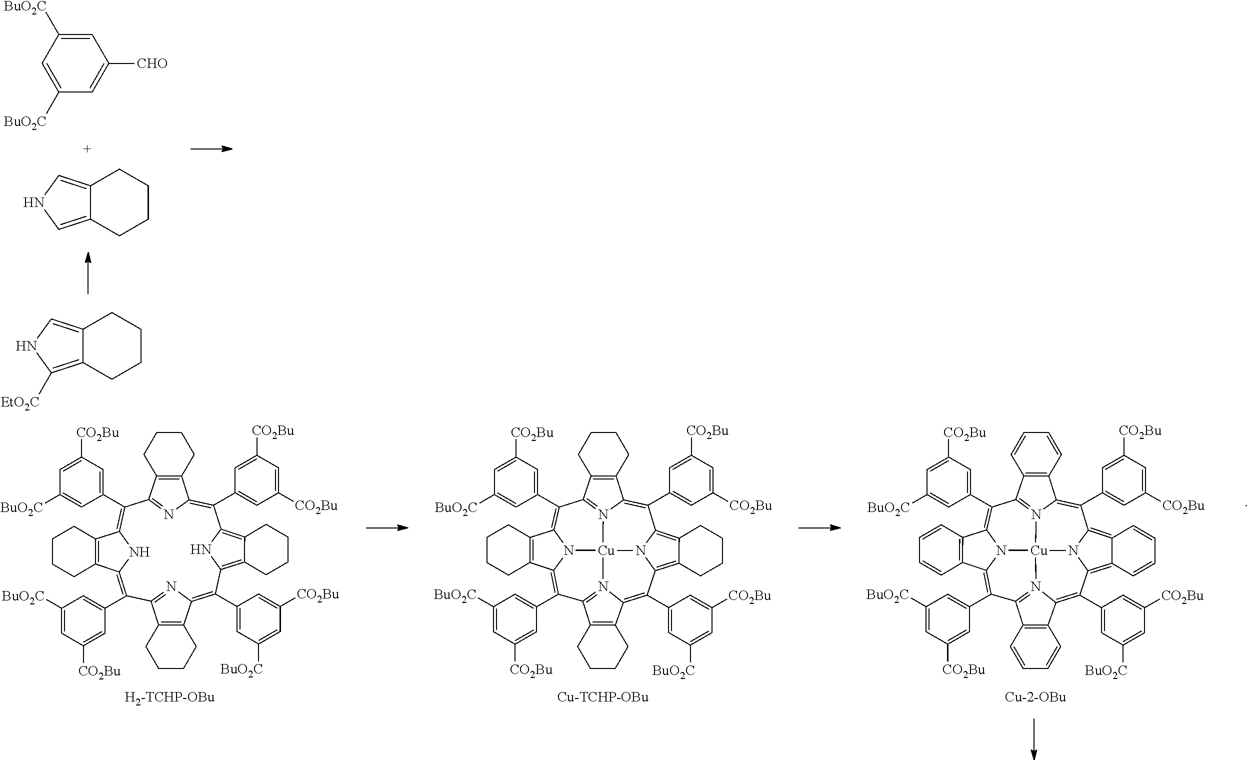

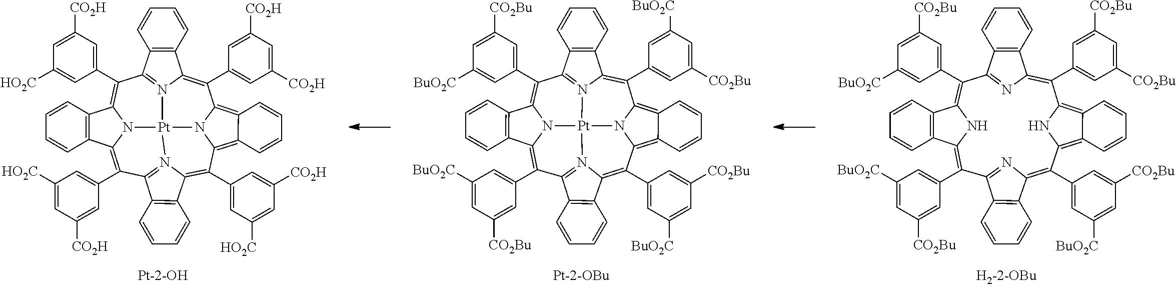

H₂-TCHP—OBu: KOH (2.60 g, 46 mmol) was added to a solution of ethyl-4,5,6,7-tetrahydro-2H-isoindole-1-carboxylate (4.20 g, 21.8 mmol) in ethylene glycol (60 ml), and the mixture was refluxed under Ar for 1 hour. The mixture was cooled to r.t. and poured into a mixture of water (100 ml) and CH₂Cl₂ (100 ml). The organic layer was separated, dried with Na₂SO₄, passed through a short silicagel column and diluted with CH₂Cl₂ to the total volume of 2 L. 3,5-dibutoxycarbonylbenzaldehyde7 (6.12 g, 4.4 mmol) was added, the solution was protected from the ambient light and bubbled with Ar for 15 min. BF₃□Et₂O (0.62 g, 4.4 mmol) was added to the mixture, and it was stirred under Ar for 2 h. DDQ (5.00 g, 22 mmol) was added, and the stirring was continued overnight. The resulting dark-green mixture was reduced in volume to 1.3 L, washed with saturated solution of Na₂SO₃ (2×250 ml), 10% Na₂SO₃ (250 ml), water (250 ml), 5% HCl (300 ml) and water (300 ml). The organic layer was dried over Na₂SO₄, and the solvent was removed in vacuum. The remaining material was chromatographed on alumina (400 g, CH₂Cl₂). The first brown and the first green bands were collected, and evaporated to dryness. The product was obtained as a brown solid. Yield: 2.84 g, 34%.

¹H NMR (CDCl₃) δ 9.05 (t, J=1.6 Hz, 4H), 9.02 (d, J=1.5 Hz, 8H), 4.48 (t, J=6.7 Hz, 16H), 2.20 (br. s, 16H), 1.85 (m, 16H), 1.54 (m, 16H), 1.44 (br s, 16H), 1.00 (t, J=7.3 Hz, 24H), −2.20 (br s, 2H).

¹³C NMR (CDCl₃+HCl, 50° C.) δ 165.6, 143.9, 140.4, 135.7, 131.8, 131.5, 125.49, 116.3, 65.9, 30.8, 25.3, 22.4, 19.4, 13.6.

MALDI-TOF (m/z): calcd. for C₁₀₀H₁₁₈N₄O₁₆: 1632.0. Found: 1632.1 [M⁺].

Cu-TCHP—OBu: H₂-TCHP—OBu (2.00 g, 1.23 mmol) and Cu(OAc)_{2□}2H₂O (2.66 g, 12.3 mmol) were dissolved in a mixture of CHCl₃ (700 ml) and MeOH (70 ml). The mixture was stirred for 2 h, then washed with 10% AcOH (100 ml), water (100 ml), saturated NaHCO₃ (100 ml) and water (100 ml) again. The organic phase was dried over Na2SO4 and evaporated to dryness. The product was obtained as a dark-red powder. Yield: 2.05 g, 99%.

MALDI-TOF (m/z): calcd. for C₁₀₀H₁₁₆CuN₄O₁₆: 1693.6. Found: 1692.8, 1693.8, 1694.8 1695.8 [M⁺; M⁺H⁺].

Cu-2-OBu: Cu-TCHP—OBu (2.00 g, 1.18 mmol) and DDQ (4.54 g, 20 mmol) were dissolved in 150 ml of dry THF and refluxed for 40 min. After cooling to r.t., the mixture was diluted with CH₂Cl₂ (200 ml) and washed with 10% Na₂SO₃ (2×100 ml), 10% Na₂CO₃ (100 ml) and water (100 ml). The organic phase was dried over Na₂SO₄ and evaporated to dryness. The residue underwent chromatography on silica gel 100-200 mesh (100 g, CH₂Cl₂), and the first green band was collected. The product was isolated as a deep green powder. Yield: 0.96 g, 46%.

MALDI-TOF (m/z): calcd. for C₁₀₀H₁₀₀CuN₄O₁₆: 1677.4. Found: 1676.7, 1677.7, 1678.7, 1679.7 [M⁺; M⁺H⁺].

H2-2-OBu: Cu-2-OBu (50 mg, 0.029 mmol) was dissolved in H₂SO₄ conc. (200 ml) and immediately poured onto crushed ice (300 g). The resulting solution was extracted with CH₂Cl₂ (2×200 ml), the organic phase was washed with water (200 ml), saturated NaHCO₃ (200 ml) and dried over Na₂SO₄. The solvent was removed in vacuum, and the product was purified on a short silica gel column (CH₂Cl₂-THF, 5:1). The product was obtained as a green solid. Yield: 37 mg, 77%.

¹H NMR (CDCl₃, 45° C.) δ 9.27 (t, J=1.5 Hz, 4H), 9.20 (d, J=1.5 Hz, 8H), 7.25 (br s, 8H), 7.07 (br s, 8H), 4.45 (t, J=6.7 Hz, 16H), 1.80 (m, 16H), 1.47 (m, 16H), 0.94 (t, J=7.3 Hz, 24H).

¹³C NMR (CDCl₃, 45° C.) δ 165.8, 142.5, 139.0, 132.1, 131.2, 126.6, 124.3, 114.1, 65.7, 30.8, 19.3, 13.7.

MALDI-TOF (m/z): calculated for C₁₀₀H₁₀₂N₄O₁₆: 1615.9. Found: 1615.2 [M⁺].

Pt-2-OBu: Pt was inserted into H₂-2-OBu following the method described for Pt-1-OBu (vide supra) starting from PtCl₂x2PhCN (140 mg, 0.296 mmol) and H₂-2-OBu (100 mg, 0.062 mmol) in dry PhCN (50 ml). Yield: 103 mg, 92%.

¹H NMR (CDCl₃) δ 9.29 (t, J=1.5 Hz, 4H), 9.11 (d, J=1.5 Hz, 8H), 7.21 (m, 8H), 6.94 (m, 8H), 4.42 (t, J=6.7 Hz, 16H), 1.77 (m, 16H), 1.44 (m, 16H), 0.92 (t, J=7.5 Hz, 24H).

¹³C NMR (CDCl₃) δ 165.8, 142.1, 138.2, 137.3, 136.0, 132.2, 131.5, 126.1, 124.0, 116.6, 65.7, 30.6, 19.2, 13.7.

MALDI-TOF (m/z): calculated for C₁₀₀H₁₀₀N₄O₁₆Pt: 1808.9. Found: 1809.1, 1810.1, 1811.1

[M⁺H⁺].

Pt-2-OH: The butyl ester groups on Pt-2-OBu were hydrolyzed following the procedure described for Pt-1-OBu. The product was isolated as deep emerald powder. Yield: 95%.

¹H NMR (DMSO-d₆) δ 9.11 (m, 4H), 9.04 (t, J=1.3 Hz, 8H), 7.36 (m, 8H), 6.92 (m, 8H), 3.37 (br s, 8H).

¹³C NMR (DMSO-d₆) δ 167.1, 142.0, 138.3, 137.4, 136.0, 133.4, 131.8, 127.2, 124.2, 117.3.

MALDI-TOF (m/z): calculated for C₆₈H₃₆N₄O₁₆Pt: 1360.1. Found: 1359.5 [M⁺].\

### Synthesis of Pd Tetrabenzoporphyrins:

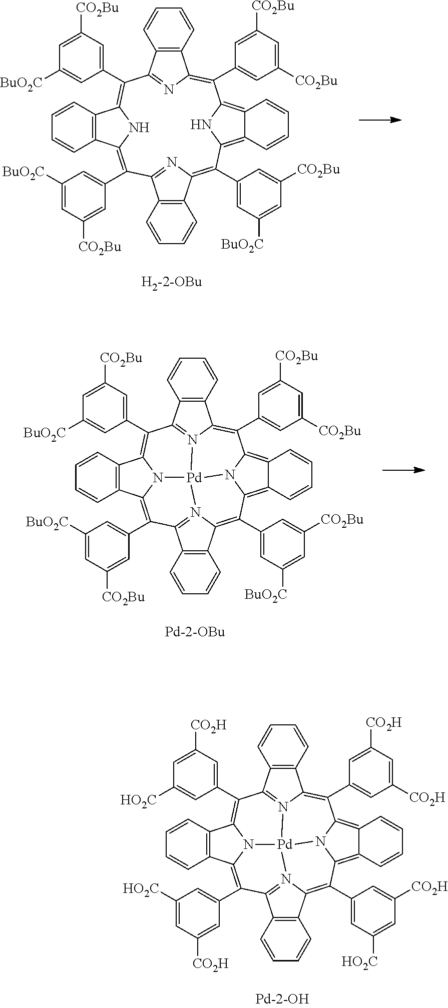

Pd-2-OBu: PdCl₂ (22 mg, 0.124 mmol) was added to a solution of H₂-2-OBu (50 mg, 0.031 mmol) in dry PhCN (20 ml), and the resulting mixture was refluxed under Ar until the conversion was complete (controlled by UV-Vis spectroscopy, typically 1 h). The mixture was evaporated to dryness, and the residual solid was purified by column chromatography on silicagel (30 g of silica, CH₂Cl₂, then CH₂Cl₂/THF 20/1). The green band was collected, the solvent was evaporated in vacuum and the residual precipitated from CH₂Cl₂ (3 ml) by addition of MeOH (12 ml). The precipitate was separated by centrifugation and dried in vacuum. Pd-2-OBu was obtained as a green powder. Yield: 50 mg, 95%.

¹H NMR (CDCl₃) δ 9.30 (t, J=1.7 Hz, 4H), 9.12 (d, J=1.7 Hz, 8H), 7.22 (m, 8H), 6.99 (m, 8H), 4.43 (t, J=6.7 Hz, 16H), 1.79 (m, 16H), 1.45 (m, 16H), 0.93 (t, J=7.5 Hz, 24H).

¹³C NMR (CDCl₃) δ 165.8, 142.4, 138.5, 138.5, 137.9, 132.3, 131.4, 126.0, 123.9, 116.4, 65.7, 30.8, 19.3, 13.7.

MALDI-TOF (m/z): calculated for C₁₀₀H₁₀₀N₄O₁₆Pd: 1720.3. Found: 1720.0, 1721.0, 1722.0, 1723.0 [M⁺; M⁺H⁺].

Pd-2-OH: The butyl ester groups on Pd-2-OBu were hydrolyzed following the procedure described for Pt-2-OBu. Yield: 95%.

¹H NMR (DMSO-d₆) δ 9.11 (t, J=1.7 Hz, 4H), 8.98 (d, J=1.7 Hz, 8H), 7.32 (m, 8H), 7.02 (m, 8H), 3.01 (br s, 8H).

¹³C NMR (DMSO-d₆) δ 165.7, 141.0, 137.2, 137.1, 136.8, 132.4, 130.3, 125.6, 122.8, 115.7.

MALDI-TOF (m/z): calculated for C₆₈H₃₆N₄O₁₆Pd: 1271.5. Found: 1271.4; 1272.4; 1273.4; 1275.4 [M⁺; M⁺H⁺].

### Synthesis of Pt Tetranaphthoporphyrins

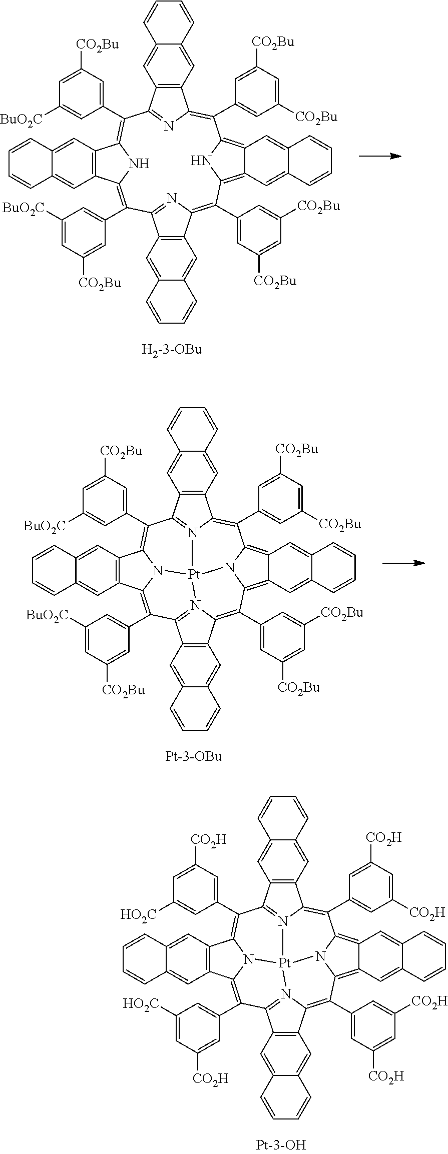

Pt-3-OBu: Pt-3-OBu was obtained as a deep green powder according to method described for Pt-1-OBu starting from PtCl₂.2PhCN (85 mg, 0.18 mmol) and H₂-3-OBu₇ (109 mg, 0.06 mmol) in dry PhCN (60 ml). Yield: 102 mg, 85%.

¹H NMR (DMF-d₇, 100° C.) δ 9.57 (t, J=1.7 Hz, 4H); 9.43 (d, J=1.7 Hz, 8H); 7.85 (m, 8H); 7.81 (br s, 8H); 7.71 (m, 8H); 4.61 (t, J=6.5 Hz, 16H), 1.91 (m, 16H); 1.55 (m, 16H); 1.02 (t, J=7.5 Hz, 24H).

¹H NMR (DMF-d₇, 100° C.) δ 9.57 (t, J=1.7 Hz, 4H); 9.43 (d, J=1.7 Hz, 8H); 7.85 (m, 8H); 7.81 (br s, 8H); 7.71 (m, 8H); 4.61 (t, J=6.5 Hz, 16H), 1.91 (m, 16H); 1.55 (m, 16H); 1.02 (t, J=7.5 Hz, 24H).

¹³C NMR (DMF-d₇, 110° C.) δ 165.8, 141.0, 139.0, 136.3, 135.6, 134.1, 131.6, 131.3, 129.2, 127.1, 121.0, 116.0, 65.9, 31.1, 19.4, 13.4.

MALDI-TOF (m/z): calcd. for C₁₁₆H₁₀₈N₄O₁₆Pt: 2009.2. Found: 2011.8 [M⁺H⁺].

Pt-3-OH: Pt-3-OBu (100 mg, 0.05 mmol) was dissolved in pyridine (30 ml) and Me₄NOH (1 ml of 1% solution in MeOH) was added to the mixture. The mixture was stirred for 10 min, the resulting green slurry was separated by centrifugation, and the solvents were removed in vacuum. Water (15 ml) was added to the remaining green solid, which dissolved immediately, forming a deep green solution. The target porphyrin-acid was precipitated upon acidification of the solution with HCl conc. The resulting green powder was washed two times with cold water by way of suspension/centrifugation and dried in vacuum. Yield: 72 mg, 92%.

¹H NMR (DMF-d₇, 80° C.) δ 9.47 (t, J=1.7 Hz, 4H), 9.28 (d, J=1.7 Hz, 8H), 7.71 (m, 8H), 7.66 (s, 8H), 7.56 (m, 8H), 3.40 (br s, 8H).

¹³C NMR (DMF-d₇, 80° C.) δ 167.2, 143.0, 139.0, 136.5, 135.8, 134.8, 132.1, 131.7, 129.5, 127.4, 124.1, 116.5.

MALDI-TOF (m/z): calculated for C₈₄H₄₄N₄O₁₆Pt: 1560.3. Found: 1562.9 [M⁺H⁺].

### Synthesis of Dendrons

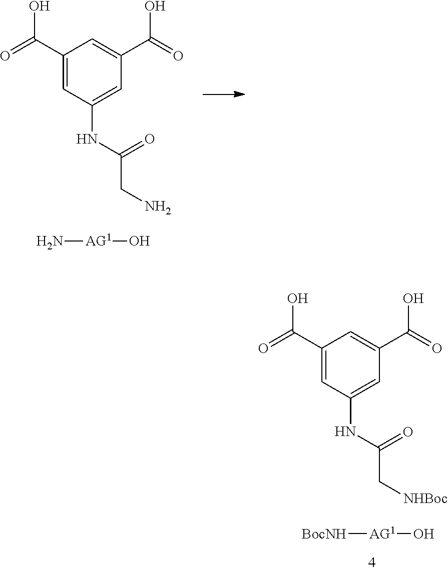

BocNH-AG1-OH (4): 3,5-Dicarboxylphenyl glycineamide (9.52 g, 40 mmol) and NaOH (3.20 g, 80 mmol) were dissolved in water (80 ml). The solution was cooled in an ice bath, and Boc₂O (9.52 g, 44 mmol) in dioxane (40 ml) was added in one portion. The resulting slurry was vigorously stirred for 2 days at room temperature, yielding a homogeneous solution. It was washed with Et₂O (100 ml), the aqueous layer was separated and acidified with citric acid (10% in water). The supernatant was decanted, and the remaining semi-solid substance completely solidified upon addition of CH₂Cl₂ (100 ml). It was filtered off, washed with water (50 ml), MeOH (50 ml) and dried in vacuum. The product was isolated as a white powder. Yield: 6.12 g, 43%.

¹H NMR (DMSO-d₆) δ 10.42 (s, 1H), 8.44 (d, J=1.2 Hz, 2H), 8.17 (t, J=1.2 Hz, 1H); 7.04 (t, J=5.4 Hz, 1H), 3.76 (d, J=6.2 Hz, 2H), 3.47 (br s, 2H), 1.41 (s, 9H).

¹³C NMR (DMSO-d6) δ 168.8, 166.4, 155.9, 139.5, 131.78, 124.5, 123.5, 78.1, 43.9, 28.2.

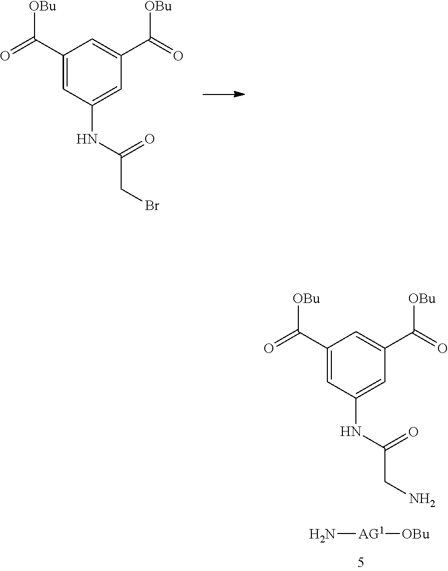

H2N-AG1-OBu (5): Dibutoxycarbonylphenyl bromoacetyleamide (20.7 g, 50 mmol) was dissolved in THF (150 ml), and the solution was added dropwise to a rapidly stirred solution of NH³ in MeOH (saturated, 500 ml) at 0° C. The mixture was kept under stirred for 4 h, and the solvent and the excess of ammonia were removed on a rotary evaporator. It is critically important to avoid heating of the reaction mixture above 30° C. Even though evaporation of the alcohol can take longer time without heating, elevated temperatures sharply decreases the yield and become purification very complicated.

The resulting yellow oil was treated with a mixture of EtOAc and hexane (1:1, 100 ml), resulting the formation of white solid, which was collected and dissolved in a mixture of water (50 ml) and EtOAc (200 ml). The product does not dissolve in water and neither does it dissolve in EtOAc; however, it readily dissolves in the mixture of water and EtOAc. The organic layer was separated, dried briefly over Na₂SO₄ (prolonged drying may lead to precipitation) and the solvent was removed in vacuum. The product was obtained in the form of a colorless foam-like solid. Yield: 12.3 g, 70%.

¹H NMR (DMSO-d₆) δ 11.03 (br s, 1H), 8.48 (d, J=1.2 Hz, 2H), 8.21 (br s, 2H), 8.14 (t, J=1.5 Hz, 1H); 4.26 (t, J=6.4 Hz, 4H), 3.86 (s, 2H), 1.66 (m, 4H), 1.39 (m, 4H), 0.90 (t, J=7.4 Hz, 6H).

¹³C NMR (DMSO-d₆) δ 165.6, 164.7, 139.2, 131.1, 124.5, 123.5, 64.9, 41.2, 30.2, 18.8, 13.6.

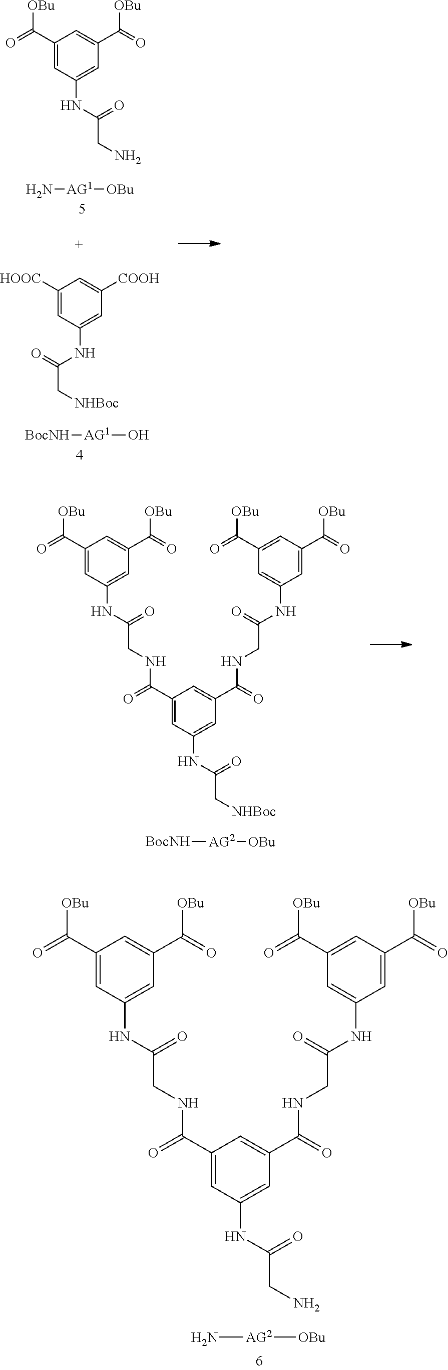

Boc.NH-AG²-OBu: Boc-protected 3,5-dicarboxylphenyl glycineamide (3.38 g, 10 mmol) and CDMT (2-chloro-4,6-dimethoxy-1,3,5-triazine) (4.38 g, 25 mmol) were dissolved in dry DMF (100 ml). The flask was sealed with rubber septa, and the solution was stirred on an ice bath for 15 min, after which NMM (N-methylmorpholine) (4.04 g, 40 mmol) was added in one portion. The resulting mixture was stirred on an ice bath for 1 h, warmed up to r.t. and stirred for 15 min. 3,5-Dibutoxycarbonylphenyl glycineamide (7.35 g, 21 mmol) was added to the resulting yellow suspension in one portion, and the mixture was stirred overnight. The mixture was poured into ice-cold water (300 ml) under vigorous stirring. The resulting precipitate was collected by filtration, washed with water (2×50 ml), rapidly washed with MeOH (50 ml) and dried in vacuum. Yield: 8.95 g, 89%.

¹H NMR (DMSO-d₆) δ 10.52 (s, 2H); 10.22 (s, 1H); 8.89 (t, J=5.7 Hz, 2H); 8.52 (s, 4H); 8.28 (s, 2H); 8.18 (s, 2H); 8.15 (s, 1H); 7.03 (t, J=5.3 Hz, 1H); 4.31 (t, J=6.5 Hz, 8H); 4.16 (d. J=5.3 Hz, 4H); 3.79 (d, J=5.2 Hz, 2H); 1.77-1.63 (m, 8H); 1.49-1.32 (m, 8H); 1.41 (s, 9H); 0.91 (t, J=7.4 Hz, 12H).

¹³C NMR (DMSO-d₆) δ 168.5; 168.2; 166.2; 164.7; 155.8; 139.6; 139.1; 134.8; 130.9; 123.9123.5; 121.0; 120.7; 78.0; 64.8; 43.8; 43.4; 30.1; 28.1; 18.6; 13.4.

MALDI-TOF (m/z): calculated for C₅₁H₆₆N₆O₁₆: 1019.1. Found: 1041.3 [M⁺Na⁺], 1057.9 [M⁺K⁺].

H₂N-AG2-OBu (6): Boc.NH-AG²-OBu (8.02 g, 8 mmol) was dissolved in trifluoroacetic acid (100 ml), the solution was kept for 1 h at r.t. and evaporated to dryness. The residual viscous oil solidified upon treatment with water (100 ml). The resulting solid was collected by filtration, washed with water (50 ml) and dried in vacuum. Yield: 7.65 g, 94%.

¹H NMR (DMSO-d₆) δ 10.78 (s, 1H), 10.59 (s, 2H), 8.93 (t, J=5.4 Hz, 2H), 8.51 (d, J=1.0 Hz, 4H), 8.28 (s, 2H), 2.26-8.21 (m, 3H), 8.18 (t, J=1.6 Hz, 2H), 4.31 30 (t, J=6.5 Hz, 8H), 4.17 30 (d, J=5.4 Hz, 4H), 3.86 (s, 2H), 1.71 (m, 8H), 1.43 (m, 8H), 0.94 (t, J=7.3 Hz, 12H).

¹³C NMR (DMSO-d₆) [symbol] 169.0, 166.8, 165.9, 165.5, 140.5, 139.1, 135.8, 131.7, 124.7, 124.2, 122.0, 121.9, 65.5, 44.1, 41.8, 30.9, 19.4, 14.2.

MALDI-TOF (m/z): calculated for C₄₆H₅₈N₆O₁₃: 903.0. Found: 922.2, 1805.5 [M⁺H₃O⁺; 2M⁺].

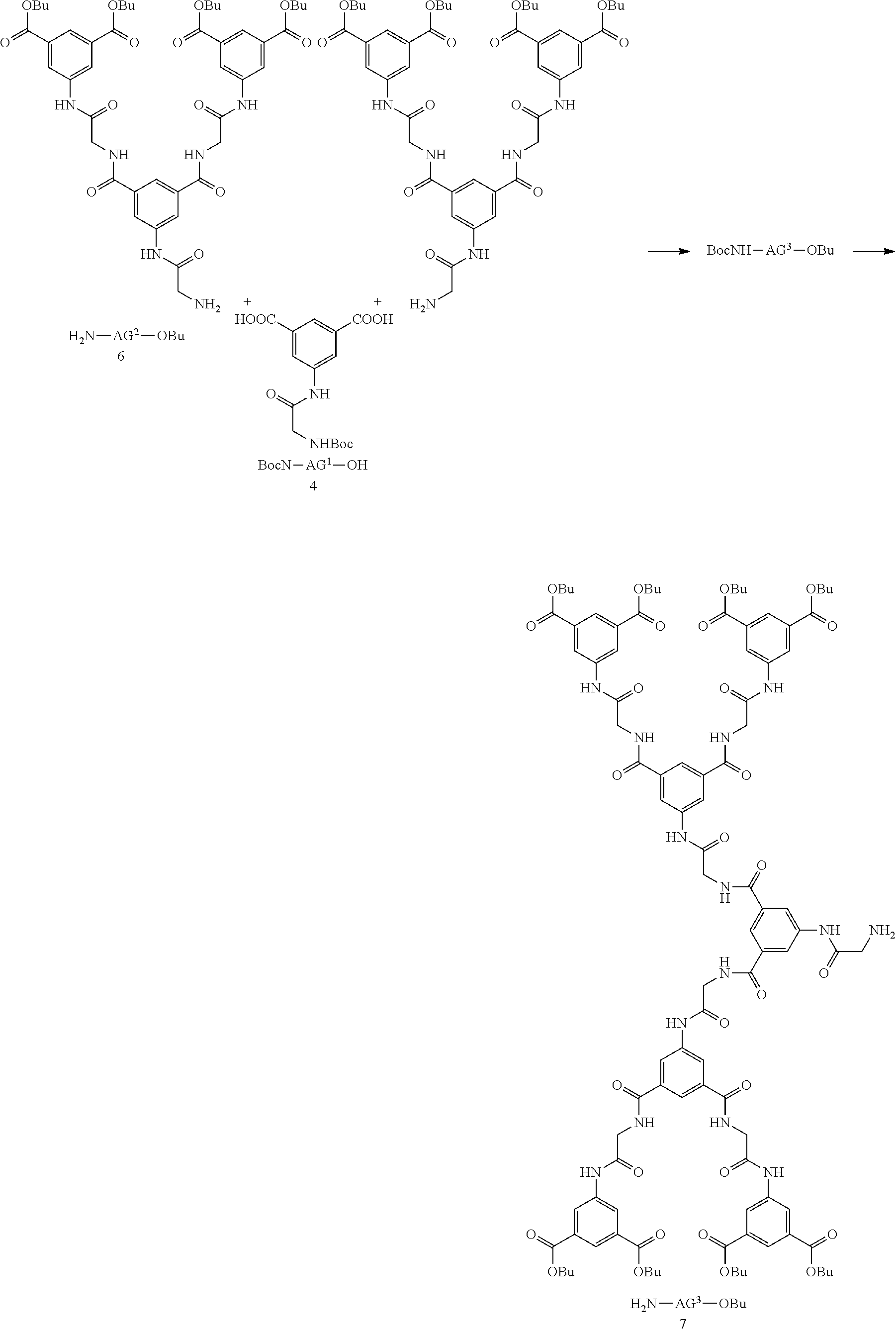

BocNH-AG³-OBu: BocNH-AG³-OBu was obtained from 5 (169 mg, 0.5 mmol) and 7 (1.03 g, 1 mmol) following the procedure described for BocNH-AG²-OBu. Yield: 1.04 g, 98%.

¹H NMR (DMSO-d₆, 110° C.) δ 10.15 (br s, 4H), 10.00 (br s, 4H), 9.81 (br s, 4H), 8.45 (d, J=1.6 Hz, 8H), 8.48-8.42 (br m, 7H), 8.26 (d, J=1.5 Hz, 4H), 8.23 (d, J=1.5 Hz, 2H), 8.18 (t, J=1.6 Hz, 4H), 8.11 (t, J=1.5 Hz, 2H), 8.10 (t, J=1.5 Hz, 1H), 4.33 (t, J=6.6 Hz, 16H), 4.18 (d, J=5.6 Hz, 8H), 4.17 (d, J=5.7 Hz, 4H), 3.79 (d, J=6.0 Hz, 2H), 1.73 (m, 161.45 (m, 16J=7.4 Hz, 24H).

¹³C NMR (DMSO-d₆, 110° C.) δ 169.22, 169.18, 168.9, 168.7, 167.1, 167.1, 165.6, 140.4, 139.8, 136.0, 136.0, 132.0, 124.9, 124.7, 122.3, 122.2, 121.7, 121.6, 79.1, 65.5, 46.3, 45.2, 44.4, 31.0, 28.9, 19.3, 13.9.

NH₂-AG³-OBu (7): was obtained from BocNH-AG³-OBu (1.00 g, 0.5 mmol) following the procedure described for the synthesis of 6. Yield: 1.00 g, 99%.

¹H NMR (DMSO-d₆) δ 10.61 (s, 1H), 10.44 (s, 4H), 10.32 (s, 2H), 8.78 (s, 4H), 8.49 (d, J=1.5 Hz, 8H), 8.28 (d, J=1.3 Hz, 4H), 8.23 (d, J=1.2 Hz, 2H), 8.19 (t, J=1.2 Hz, 1H), 8.16 (t, J=1.5 Hz, 4H), 8.14 (t, J=1.3 Hz, 2H), 4.86 (br s, 2H), 4.30 (t, J=6.5 Hz, 16H), 4.20-4.10 (m, 12H), 3.80 (br s, 2H), 1.70 (m, 16H), 1.42 (m, 16H), 0.93 (t, J=7.3 Hz, 24H).

¹³C NMR (DMSO-d₆) δ 168.8, 168.5, 166.8, 166.7, 165.3, 165.0, 140.3, 139.6, 138.8, 135.8, 135.4, 131.5, 124.5, 124.0, 123.1, 121.9, 121.7, 121.3, 65.3, 44.0, 41.6, 41.00, 30.7, 19.2, 14.00.

MALDI-TOF (m/z): calculated for C₁₀₂H₁₂₂N₁₄O₂₉: 2008.0. Found: 2030.6 [M⁺Na⁺]; 2047.6 [M⁺Na⁺]. Each peak was accompanied by a satellite with mass incremented by 552 units.

### Synthesis of Porphyrin-Dendrimers

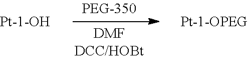

Pt-1-OPEG: was synthesized following the procedure described for 8. Yield: 28%.

¹H NMR (DMSO-d₆) δ 8.99 (t, J=1.5 Hz, 4H), 8.93 (d, J=1.5 Hz, 8H), 8.77 (s, 8H), 4.6-3.0 (multiple peaks, ˜270H).

¹³C NMR (DMSO-d₆) δ 164.6, 140.8, 140.2, 136.9, 130.8, 129.5, 129.3, 120.0, 71.0, 69.7, 69.6, 69.5, 69.5, 69.4, 68.0, 64.3, 30.1.

MALDI-TOF (m/z): calculated for C₁₇₂H₂₆₈N₄O₇₂Pt: 3739.0. Found: series of peaks with mass increment 44, normally distributed around 3778 Da. [M⁺K⁺].

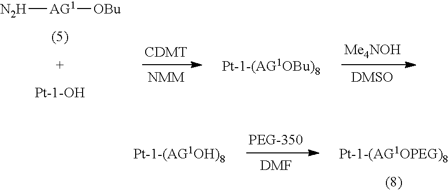

Pt-1-(AG¹OBu)₈: Pt-1-OH (46.4 mg, 0.04 mmol) and CDMT (2-chloro-4,6-dimethoxy-1,3,5-triazine) (0.07 g, 0.4 mmol) were dissolved in dry DMF (10 ml). The flask was sealed with rubber septa, and the solution was stirred on an ice bath for 15 min. NMM (N-methylmorpholine) (0.08 g, 0.8 mmol) was added in one portion, the resulting mixture was stirred on an ice bath for 1 h, warmed up to r.t. and stirred for additional 15 min. 3,5-Dibutoxycarbonylphenyl glycineamide (140 mg, 0.4 mmol) was added to the resulting orange suspension in one portion, and the mixture was stirred overnight. It was poured into ice-cold water (20 ml), the precipitate was separated by centrifugation, washed with water (20 ml) and MeOH (20 ml) by suspension/centrifugation cycles and dried in vacuum. Pt-1-(AG¹OBu)₈ was isolated as an orange powder. Yield: 130 mg, 85%.

¹H NMR (DMSO-d₆) δ 10.45 (s, 8H), 9.23 (t, J=5.7 Hz, 8H), 8.97 (t, J=1.5 Hz, 4H), 8.90 (d, J=1.5 Hz, 8H), 8.87 (s, 8H), 8.47 (d, J=1.5 Hz, 16H), 8.12 (t, J=1.6 Hz, 8H), 4.24 (t, J=6.5 Hz, 32H), 4.17 (d, J=5.3 Hz, 16H), 1.64 (m, 32H), 1.36 (m, 32H), 0.87 (t, J=7.4 Hz, 48H).

¹³C NMR (DMSO-d₆) δ 168.9, 167.0, 165.6, 141.4, 141.3, 140.4, 135.0, 134.1, 131.99, 131.95, 127.5, 124.9, 124.7, 122.0, 65.5, 44.6, 30.9, 19.3, 13.9.

MALDI-TOF (m/z): calculated for C₁₉₆H₂₂₀N₂₀O₄₈Pt: 3819.0. Found: 3819.0, 3841.0 [M⁺; M⁺Na⁺].

Pt-1-(AG¹-OH)₁: Pt-1-(AG¹-OBu)₈ (106 mg, 0.0277 mmol) was dissolved in DMSO (25 ml) and Me₄NOH (0.1 ml of 25% in MeOH) was added in one portion. The mixture was stirred for 20 min, diluted with water (25 ml) and acidified with HCl conc. The resulting suspension was centrifuged, the precipitate washed with water (20 ml), dissolved in NaOH aq. (pH˜9), and left overnight. The solution was acidified with 0.1 HCl aq., the resulting suspension was centrifuged, the precipitate washed with water and dried in vacuum. Pt-1-(AG¹-OH)₈ was isolated as an orange powder. Yield: 72 mg, 89%.

¹H NMR (DMSO-d₆) δ 10.41 (s, 8H), 9.37 (s, 8H), 8.98 (s, 4H), 8.95 (s, 8H), 8.90 (s, 8H), 8.41 (s, 16H), 8.13 (s, 8H), 4.16 (s, 16H).

¹³C NMR (DMSO-d₆) δ 168.2, 167.1, 166.1, 140.5, 140.4, 139.1, 134.5, 133.1, 132.8, 131.6, 127.0, 124.7, 123.5, 121.4, 43.7.

MALDI-TOF (m/z): calculated for C132H92N20O48Pt: 2921.3. Found: 3032.2 (C₁₃₂H₈₇N₂₀O₄₈PtNa₅+H⁺); 3048.4 (C₁₃₂H₈₇N₂₀O₄₈PtNa₄K+H⁺); 3060.5 (C₁₃₂H₈₈N₂₀O₄₈PtNa₂K₂+H₃O⁺); 3076.0 (C₁₃₂H₈₅N₂₀O₄₈PtNa₇+H⁺); 3090.3 (C₁₃₂H₈₆N₂₀O₄₈PtNa₆⁺H₂O+H₃O⁺); 3098.4 (C₁₃₂H₈₅N₂₀O₄₈PtNa₇+Na⁺).

Pt-1-(AG¹-OPEG)₈ (8): Pt-1-(AG¹-OH)₈ (73 mg, 0.025 mmol), DCC (dicyclohexylcarbodiimide) (0.206 g, 1 mmol), HOBt (1-hydroxybenzotriazole) (0.135 g, 1 mmol) were dissolved in dry DMF (1 ml), PEG350 (2 ml) and three drops of sym-collidine were added to the mixture, and it was left to react overnight. The resulting red mixture was poured into water (20 ml) and extracted with CH₂Cl₂ (2×20 ml). The organic phase was dried over Na₂SO₄ and evaporated in vacuum. The resulting viscous liquid was diluted with THF (2 ml), and Et₂O (25 ml) was added to yield a red oily precipitate, containing the target compound together with unreacted PEG350. The precipitate was collected by centrifugation. This dissolution-precipitation procedure was repeated three times. The product was obtained as a red viscous solid. Yield: 130 mg, ˜60%.

¹H NMR (DMSO-d₆) δ 10.68 (s, 8H), 9.38 (s, 8H), 9.01 (s, 4H), 8.93 (s, 8H), 8.90 (s, 8H), 8.53 (s, 8H), 8.16 (s, 8H), 4.5-3.0 (multiple peaks, ˜500H).

¹³C NMR (DMSO-d₆) δ 168.0, 165.6, 164.3, 140.0, 139.4, 138.9, 133.9, 132.6, 131.1, 130.3, 126.6, 123.7, 123.3, 120.9, 70.8, 69.3, 67.8, 64.1, 57.5, 43.2.

MALDI-TOF spectrum showed two bell-shaped peaks centered around 8.0 and 15.2 kDa. Calculated MWav. 8.2 kDa.

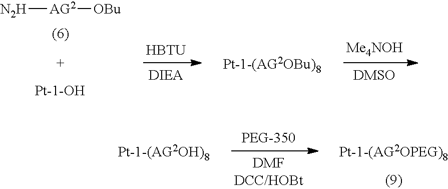

Pt-1-(AG²OBu)₈: Pt-1-OH (11.6 mg, 0.01 mmol) was dissolved in dry NMP (10 ml) at 140° C. during 10 min. Longer heating may lead to decomposition of the porphyrin. The solution was cooled to room temperature, 2-(1H-benzotriazol-1-yl)-1,1,3,3-tetramethyluronium hexafluorophosphate (HBTU) (0.1 mmol, 38 mg) was added, and the mixture was stirred for 5 min. N,N-diisopropylethylamine (DIEA) (65 mg, 0.5 mmol) was added to the mixture in one portion by a syringe. This was immediately followed by addition of 6 (0.11 mmol, 110 mg) in dry NMP (2 ml), and the mixture was left overnight under stirring. The mixture was poured into aq. NaCl (3%, 20 ml), and the resulting precipitate was collected by centrifugation and washed with water (2×20 ml), MeOH (2×20 ml), and Et₂O by repetitive suspension/centrifugation. The crude product was dissolved in NMP (20 ml), and isothiocyanate immobilized on cross-linked polystyrene (Aldrich) was added (50 mg, 0.05 mmol). The mixture was stirred overnight. The resin was filtered trough a cotton clot, washed with NMP (2×10 ml), and the combined solutions containing the coupling product were poured into aq. NaCl (3%, 40 ml). The precipitate formed was collected by centrifugation and washed with water (2×20 ml), MeOH (2×20 ml), and Et₂O by repetitive suspension/centrifugation. The target porphyrin-dendrimer was isolated as red solid. Yield: 74 mg, 90%.

Thus prepared compound contained ca. 10% of impurities (by mass). Further purification was performed by size exclusion chromatography on polystyrene beads (column: 100×5 cm, mobile phase—THF), collecting the front edge of the color band. Thus purified compound was obtained in 5-10% yield.

¹H NMR (DMSO-d₆, 100° C.) δ 10.16 (s, 16H), 10.08 (s, 8H), 8.96 (br s, 4H), 8.94 (br s, 8H), 8.85 (d, J=1.5 Hz, 8H), 8.84 (s, 8H), 8.47 (t, J=5.2 Hz, 16H), 8.42 (d, J=1.6 Hz, 32H), 8.25 (d, J=1.5 Hz, 16H), 8.14 (t, J=1.6 Hz, 16H), 8.09 (t, J=1.5 Hz, 8H), 4.27 (t, J=6.5 Hz, 64H), 4.20 (d, J=5.9 Hz, 16H), 4.12 (d, J=5.7 Hz, 32H), 1.67 (m, 64H), 1.40 (m, 64H), 0.90 (t, J=7.5 Hz, 96H).

¹³C NMR (DMSO-d₆, 100° C.) δ 167.8, 167.6, 165.9, 165.8, 164.4, 140.2, 140.1, 139.3, 138.7, 134.7, 134.0, 133.0, 130.9, 130.7, 126.3, 123.7, 123.4, 121.2, 120.9, 120.5, 64.4, 43.3, 43.2, 29.8, 18.2, 12.9.

MALDI-TOF (m/z): calculated for C420H476N520112Pt: 8239.6. Found: 8240.4 [M⁺+]; 8282.4 [M+H₂O+Na⁺]. Each ion was accompanied by satellites with masses incremented by 554 mass units. The Intensities of the satellite peaks was dependent on instrument parameters (laser intensity, voltage).

NOTE: In the early experiments, before the coupling conditions were established, MALDI analyses of reaction mixtures revealed multiple peaks. The ratio between these peaks was found to be independent of the ionization power and of the method used for the sample preparation, which suggested that the distribution was caused by the presence of imperfect dendrimers and not the fragmentation. In order to confirm this hypothesis, two model experiments were performed. The synthesis was carried out in the presence of either 2-fold excess of G2 dendron 7 or 2-fold excess of the core porphyrin (Pd-3-OH). The MALDI analysis showed two predictably different product distributions: in the case of the dendron excess—the distribution was enriched by peaks of higher molecular weight; whereas in the case of the excess of the porphyrin, peaks of lower molecular weight were predominant. Importantly, both distributions consisted of the peaks with the same masses, and only their ratios were different. It was speculated that the synthesis in which the porphyrin was used in excess resulted in a mixture of imperfect dendrimers. The fact that exactly the same peaks were observed in the experiment where the dendron was present in excess; suggests that the cause of the peak distribution (as opposed to the single peak, corresponding to octasubstituted porphyrindendrimer), was the incomplete reaction and not the ion fragmentation.

Pt-1-(AG²OH)₈: Pt-1-(AG²OH)₈ was obtained following the procedure described for Pt-1-(AG¹OH)₈, starting from crude Pt-1-(AG¹OH)₈, not treated with amine scavenging resin and not purified by chromatography. Yield: 208 mg, 81% (for coupling and hydrolysis combined).

¹H NMR (DMSO-d₆, 100° C.) δ 10.17 (s, 8H), 10.13 (s, 16H), 9.01 (t, J=5.4 Hz, 4H), 8.97 (broad s, 8H), 8.86 (d, J=5.4 Hz, 8H), 8.55 (t, J=4.3 Hz, 16H), 8.43 (s, 8H), 8.41 (s, 32H), 8.27 (s, 16H), 8.17 (s, 16H), 8.10 (s, 8H), broad 5.92 (s, 32H), 4.22 (d, J=4.3 Hz, 16H), 4.13 (t, J=5.4 Hz, 32H).

¹³C NMR (DMSO-d₆, 100° C.) δ 167.6, 167.5, 166.7, 166.0, 165.8, 140.1, 140.0, 138.9, 138.7, 134.7, 134.0, 133.0, 131.7, 130.9, 126.2, 124.3, 123.5, 121.1, 120.5, 115.2, 46.7, 43.3.

MALDI-TOF (m/z): calculated for C₂₉₂H₂₂₀N₅₂O₁₁₂Pt: 6444.2. Found: broad peak centered around 6466 [M+Na⁺], accompanied by series of peaks with mass increment ˜450. Intensity of satellites depends on instrument parameters.

Pt-1-(AG²-OPEG)₈ (9): Pt-1-(AG²-OPEG)₈ was obtained from Pt-1-(AG²OH)₈ (50 mg, 0.0078 mmol) following the procedure described for 8. Yield: 75 mg, ˜60%.

¹H NMR (DMSO-d₆) δ 10.32 (bs, 16H), 10.21 (bs, 8H), 9.05 (bs, 8H), 8.97 (bs, 4H), 8.87 (bs 16H), 8.59 (bs, 16H), 8.48 (bs, 32H), 8.29 (s, 16H), 8.17 (bs, 16H), 8.13 (bs, 8H), 4.5-3.0 (mult. overl. sign., ˜1060H).

¹³C NMR (DMSO-d₆) δ 167.8, 167.5, 165.9, 165.8, 164.4, 140.2, 139.4, 138.8, 134.7, 134.0, 131.0, 130.9, 130.5, 123.8, 123.6, 121.6, 121.0, 120.4, 70.9, 69.6, 69.5, 69.4, 69.2, 67.9, 67.9, 64.0, 57.6, 43.4, 43.2.

MALDI-TOF spectrum showed three broad bell-shaped peaks centered around 16.6; 33.2 and 49.8 kDa. Calculated MWav. 17.1 kDa.

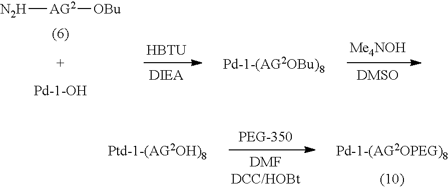

The above reaction sequence was performed following the procedures described for Pt-1-AG²-OBu/OH/OPEG (vide supra).

Pd-1-(AG²OH)₈: Yield: 195 mg, 76% (for coupling and hydrolysis combined). ¹H NMR (DMSO-d₆) δ 10.43 (bs, 24H), 9.35 (bs, 8H), 8.97 (broad s, 4H), 8.92 (bs, 32H), 8.43 (s, 32H), 8.27 (s, 16H), 8.13 (s, 16H), 8.10 (s, 8H), 4.17 (bs, 16H), 4.08 (bs, 32H).

Pd-1-(AG²-OPEG)₈ (10): Yield: 373 mg, ˜72%. ¹H NMR (DMSO-d₆) δ 10.55 (bs, 16H), 10.44 (bs, 8H), 9.34 (bs, 8H), 8.98 (bs, 4H), 8.89 (bs 32H), 8.49 (bs, 32H), 8.29 (s, 16H), 8.14 (bs, 24H), 4.5-3.0 (mult. overl. sign., ˜1060H). MALDI-TOF spectrum showed broad bell-shaped peak centered around 16.2. Calculated MWav. 17.1 kDa.

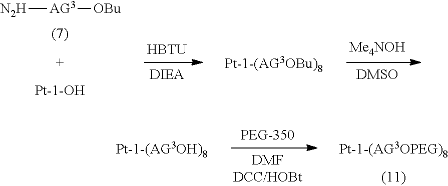

The above reaction sequence was performed following the procedures described for Pt-1-AG²-OBu/OH/OPEG (vide supra).

Pt-1-(AG³OBu)₈: Yield: 498 mg, 73%. MALDI-TOF (m/z): calculated for C₈₆₈H₉₈₈N₁₁₆O₂₄₀Pt: 17080.8. Found: 15095 [Pt-1-(AG³-OBu)₇+Na⁺]+17103 [M+Na⁺]. Each peak is accompanied by set of at least eight peaks with mass increments of 551 units. The peaks are centered around mass of 16 kDa.

Pt-1-(AG³OH)₈: Yield: 351 mg, 65% (for coupling and hydrolysis combined!) Because of the high sample viscosity and low mobility of the dendritic branches, it was impossible to obtain a high resolution NMR spectrum even at elevated temperature. Nevertheless, the ratio of the peak intensities was in a good agreement with the expected molecular formula. ¹H NMR (DMSO-d₆, 100° C.) δ 10.45 (bs, 52H), 9.5-7.9 (series of overlapped peaks, 228H), 4.1-3.8 (series of overlapped peaks, 112H). ¹³C NMR (DMSO-d₆) δ 167.8, 167.6, 166.0, 165.9, 165.8, 139.1, 138.7, 134.5, 131.3, 124.2, 123.2, 120.6, 120.5, 43.1, 42.9 (all peaks are broadened). MALDI-TOF (m/z): calculated for C₆₁₂H₄₇₆N₁₁₆O₂₄₀Pt: 13490.0. Found: broad bell-shaped peak centered at 13.5 kDa.

Pt-1-(AG³OPEG)₈ (11): 100 mg of Pt-1-(AG³OH)₈ yield ˜140 mg of Pt-1-(AG³OH)₈. Yield: ˜55%. ¹H NMR (DMSO-d₆) δ 10.7-10.3 (set of broad overlapping peaks, 52H), 9.5-7.9 (set of broad overlapping peaks, 228H), 4.6-3.0 (set of broad overlapping peaks, ˜2400H). ¹³C NMR (DMSO-d₆) 168.3, 168.0, 166.3, 165.2, 164.7, 139.9, 139.2, 134.9, 130.7, 124.9, 124.1, 123.7, 121.1, 71.2, 69.7, 69.5, 68.2, 67.0, 64.5, 58.0, 43.4, 43.2. MALDI-TOF spectrum showed a very broad low-intensity peak centered at 31.1 kDa. Calculated MWav. 33.7 kDa.

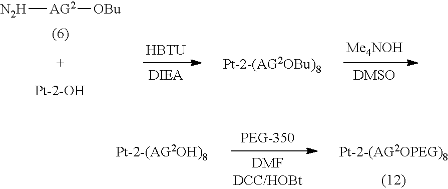

The above reaction sequence was performed following the procedures described for Pt-1-AG²-OBu/OH/OPEG (vide supra).

Pt-2-(AG²OBu)₈: Yield: 293 mg, 87% (prior to chromatographic purification). ¹H NMR (DMSO-d₆, 100° C.) δ 10.16 (s, 16H), 10.12 (s, 8H), 9.16 (br s, 4H), 9.02 (t, J=6.0 Hz, 8H), 8.96 (s, 8H), 8.46 (t, J=6.0 Hz, 16H), 8.42 (d, J=1.4 Hz, 32H), 8.26 (d, J=1.0 Hz, 16H), 8.14 (t, J=1.6 Hz, 16H), 8.09 (t, J=1.4 Hz, 8H), 7.34 (m, 8H), 7.08 (m, 8H), 4.27 (t, J=6.4 Hz, 64H), 4.20 (d, J=4.9 Hz, 16H), 4.12 (d, J=5.6 Hz, 32H), 1.67 (m, 64H), 1.40 (m, 64H), 0.90 (t, J=7.5 Hz, 96H).

¹³C NMR (DMSO-d₆, 100° C.) δ 167.8, 167.5, 165.9, 165.4, 164.4, 140.5, 139.3, 138.7, 136.5, 135.2, 134.7, 133.9, 130.7, 130.5, 127.6, 126.0, 123.7, 123.4, 123.1, 121.0, 120.5, 116.8, 64.4, 43.3, 43.2, 29.8, 18.2, 12.9.

MALDI-TOF (m/z): calculated for C₄₃₆H₄₈₄N₅₂O₁₁₂Pt: 8439.9. Found: 8433.9 [M+H⁺], 8462.2 [M+Na⁺], 8478.8 [M+K⁺]. Each ion is accompanied by a series of satellites with mass increments of 551 or 1102 mass units. The intensity of the satellite peaks were found to be dependent on the instrument parameters.

Pt-2-(AG²OH)₈: Yield 215 mg, 81% (for coupling and hydrolysis combined!). ¹H NMR (DMSO-d₆, 80° C.) δ 10.24 (s, 8H), 10.19 (s, 16H), 9.17 (s, 8H), 9.15 (s, 4H), 8.98 (s, 8H), 8.61 (s, 16H), 8.41 (s, 32H), 8.27 (s, 16H), 8.16 (s, 16H), 8.11 (s, 8H), 7.37 (s, 8H), 7.08 (s, 8H), 4.20 (s, 16H), 4.13 (s, 32H).

Pt-2-(AG²OPEG)₈ (12): Pt-2-(AG²OPEG)₈ was obtained from Pt-2-(AG²-OH)₈ (50 mg, 0.0078 mmol) following the procedure described for 8. Yield: 75 mg, ˜60%. ¹H NMR (DMSO-d₆) δ 10.65-10.40 (m, 24H), 9.42 (bs, 8H), 9.20 (bs, 4H), 9.02 (bs 8H), 8.91 (bs, 16H), 8.49 (bs, 32H), 8.29 (s, 16H), 8.17 (bs, 24H), 7.40 (m, 8H), 7.02 (m, 8H), 4.5-3.0 (mult. overl. sign., ˜1140H).

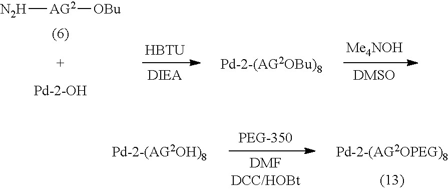

The above reaction sequence was performed following the procedures described for Pt-1-AG²-OBu/OH/OPEG (vide infra).

Pd-2-(AG²OBu)₈: Yield 307 mg, 92% (before chromatographic purification).

¹H NMR (DMSO-d₆, 100° C.) δ 10.25 (s, 16H), 10.21 (s, 8H), 9.17 (bs, 4H), 9.12 (bs, 8H), 8.97 (s, 8H), 8.50 (t, J=5.5 Hz, 16H), 8.44 (d, J=1.5 Hz, 32H), 8.27 (d, J=1.0 Hz, 16H), 8.13 (t, J=1.6 Hz, 16H), 8.10 (t, J=1.4 Hz, 8H), 7.35 (m, 8H), 7.10 (m, 8H), 4.27 (t, J=6.4 Hz, 64H), 4.20 (d, J=4.9 Hz, 16H), 4.12 (d, J=5.6 Hz, 32H), 1.67 (m, 64H), 1.40 (m, 64H), 0.90 (t, J=7.5 Hz, 96H).

¹³C NMR (DMSO-d₆, 100° C.) δ 167.8, 167.5, 165.9, 165.4, 164.4, 139.3, 138.7, 137.4, 136.9, 135.1, 134.7, 134.0, 131.0, 130.7, 127.6, 125.8, 123.7, 123.3, 121.0, 120.6, 120.5, 118.4, 64.4, 43.25, 43.2, 39.5, 29.8, 18.2, 12.9.

MALDI-TOF (m/z): calculated for C₄₃₆H₄₈₄N₅₂O₁₁₂Pd: 8351.2. Found: 8374.5 [M+Na⁺]. The MI is accompanied by a set of satellites with masses incremented by 551, 1102, 1653 and 2204 mass units. The intensities of the satellites were found to be dependent on the instrument parameters.

Pd-2-(AG²OH)₈: Yield 207 mg, 79% (for coupling and hydrolysis combined!)

¹H NMR (DMSO-d₆, 80° C.) δ 10.22 (s, 8H), 10.18 (s, 16H), 9.13 (bs, 4H), 9.05 (bs, 8H), 8.97 (bs, 8H), 8.59 (s, 16H), 8.41 (s, 32H), 8.27 (s, 16H), 8.15 (s, 16H), 8.13 (s, 8H), 7.38 (s, 8H), 7.11 (s, 8H), 4.21 (s, 16H), 4.13 (s, 32H).

¹³C NMR (DMSO-d₆, 80° C.) δ 167.7, 167.6, 167.5, 165.9, 165.4, 140.7, 139.0, 138.7, 138.6, 137.4, 136.9, 135.1, 134.7, 134.1, 131.5, 127.6, 125.9, 124.3, 123.5, 121.0, 120.5, 116.4, 43.23, 43.03.

MALDI-TOF (m/z): calculated for C₃₀₈H₂₂₈N₅₂O₁₁₂Pd: 6555.8. Found: broad peak 6657.6, [C₃₀₈H₂₂₃N₅₂O₁₁₂PdNa₅+H⁺]. The peak was accompanied by a series of satellites with mass incremented by 452 units. The intensities of the satellites were found to be dependent on the instrument parameters.

Pd-2-(AG²OPEG)₈ (13): Pd-2-(AG²OPEG)₈ was obtained from Pd-2-(AG²OH)₈ (51 mg, 0.0078 mmol) following the procedure described for 8. Yield: 75 mg, ˜60%. NMR (DMSO-d₆) δ 10.58 (bs, 16H), 10.48 (bs, 8H), 9.5-8.0 (mult. overl. sign., ˜108H), 4.5-3.0 (mult. overl. sign., ˜1106H). MALDI-TOF spectrum showed two broad bell-shaped peaks centered at 16.8 and 33.6 kDa. Calculated MWav. 17.2 kDa.

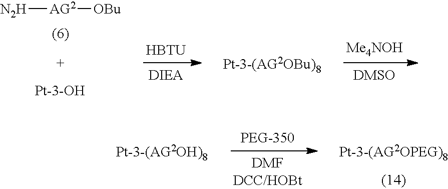

The above reaction sequence was performed following the procedures described for Pt-1-AG²-OBu/OH/OPEG respectively.

Pt-3-(AG²OBu)₈: Yield 314 mg, 91%.

¹H NMR (DMSO-d₆, 80° C.) δ 10.27 (s, 16H), 10.22 (s, 8H), 9.32 (br s, 4H), 9.17 (t, J=5.6 Hz, 8H), 9.06 (s, 8H), 8.59 (t, J=5.9 Hz, 16H), 8.45 (d, J=1.4 Hz, 32H), 8.27 (s, 16H), 8.14 (s, 16H), 8.11 (s, 8H), 7.69 (m, 8H), 7.63 (s, 8H), 7.56 (m, 8H), 4.27 (t, J=6.5 Hz, 64H), 4.19 (d, J=4.0 Hz, 16H), 4.14 (d, J=5.5 Hz, 32H), 1.67 (m, 64H), 1.40 (m, 64H), 0.90 (t, J=7.5 Hz, 96H).

¹³C NMR (DMSO-d₆, 100° C.) δ 167.8, 167.5, 165.9, 165.6, 164.4, 140.8, 139.3, 138.7, 136.1, 134.8, 134.7, 134.1, 134.0, 130.7, 130.1, 128.4, 127.6, 126.2, 123.7, 123.4, 123.0, 120.9, 120.5, 115.3, 64.3, 43.4, 43.2, 29.8, 18.2, 12.9.

MALDI-TOF (m/z): calculated for C₄₅₂H₄₉₂N₅₂O₁₁₂Pt: 8640.1. Found: 8662.3 [M₊Na⁺], 8679.3 [M+K+]. Each ion is accompanied by a set of satellites with masses incremented by 551 and 1102 units. The intensities of the satellites were found to be dependent on the instrument parameters.

Pt-3-(AG²OH): Yield 205 mg, 75% (coupling and hydrolysis combined!) ¹H NMR (DMSO-d₆, 100° C.) δ 10.21 (s, 8H), 10.18 (s, 16H), 9.30 (bs, 4H), 9.16 (bs, 8H), 9.04 (bs, 8H), 8.59 (s, 16H), 8.41 (s, 32H), 8.26 (s, 16H), 8.15 (s, 16H), 8.10 (s, 8H), 7.69 (m, 8H), 7.61 (s, 8H), 7.56 (m, 8H), 4.19 (s, 16H), 4.12 (s, 32H). ¹³C NMR (DMSO-d6, 80° C.) δ 167.7, 167.5, 167.2, 165.9, 165.7, 140.8, 139.0, 138.8, 136.1, 134.8, 134.1, 134.1, 131.5, 130.1, 128.4, 127.7, 126.4, 124.3, 123.5, 123.0, 120.9, 120.6, 120.5, 115.4, 43.30, 43.35.

Pt-3-(AG²OPEG)₈ (14): Pt-3-(AG²OPEG)₈ was obtained from Pt-3-(AG²OH)₈ (50 mg, 0.0078 mmol) following the procedure described for 8. Yield: 85 mg, ˜65%. ¹H NMR (DMSO-d₆) δ 10.55 (bs, 16H), 10.48 (bs, 8H), 9.5-7.9 (mult. overl. sign., ˜116H), 7.75-7.50 (m, 16H), 4.5-3.0 (mult. overl. sign., ˜1050H).

### Example 3

### Probe Components

### Phosphorescent Chromophores

Relatively few chromophores exhibit bright phosphorescence at ambient temperatures. Among them, α-diimine complexes of Ru, Ir and some other transition metals, cyclometallated complexes of Ir and Pt and Pt and Pd complexes of porphyrins and related tetrapyrroles have been used in oxygen sensing, although some other systems have also been proposed.

For tissue applications, it is desirable that probes posses absorption bands in the near-infrared region (NIR). It has been shown that lateral π-extension of Pt and Pd porphyrins by annealing their pyrrole residues with external aromatic rings renders chromophores with dramatically red-shifted absorption bands and strong room-temperature phosphorescence. Structures and absorption and emission spectra of Pd tetraarylporphyrin (PdP), Pd tetraaryltetrabenzoporphyrin (PdTBP) and Pd tetraaryltetranaphthoporphyrin (PdTNP), are shown in FIG. 8. Spectra of Pt complexes are very similar in shape but slightly blue-shifted (10-15 nm) compared to the Pd counterparts. The three basic porphyrin types, P, TBP and TNP, are designated in FIG. 8 as 1, 2 and 3, respectively. In this study, we used mesotetraarylporphyrins, where Ar=3,5—RO₂C—C₆H. (To distinguish between different groups R and metals (Pt and Pd), prefixes and endings are added to the numbers indicating the porphyrin types. For example, Pt complex of TBP with butoxycarbonyl substituents is abbreviated as Pt-2-OBu.)

In Pt and Pd porphyrins, S1→T1 intersystem crossing is the predominant pathway of deactivation of the singlet excited states (S1), and the resulting triplet states are typically highly emissive (phosphorescent). In regular (non-extended) porphyrins macrocycle deviations from planarity dramatically enhance the competing non-radiative triplet decay and quench the phosphorescence. In contrast, Pt and Pd meso-tetraarylaryl tetrabenzoporphyrins (TBP's), although highly non-planar, phosphoresce with high quantum yields. Pd and Pt tetraaryltetranaphthoporphyrins (FIG. 8 and Example 1) are also non-planar and in addition have much narrower T1-S0 gaps (Table I). Nevertheless they still phosphoresce, although weaker than TBP's and regular porphyrins. The emission spectra in FIG. 7b are scaled to reflect the relative phosphorescence quantum yields, and the complete photophysical data for the chromophores are summarized in Table II. Taken together, the absorption bands of Pt and Pd P's, TBP's and TNP's cover practically the entire UV-vis-NIR range, presenting multiple opportunities for excitation. The absorption Qbands of TBP's and TNP's are shifted to the red, i.e. into the region between ˜630 and ˜950 nm, where the absorption of endogenous chromophores is significantly lower. The oscillator strengths of these bands are extremely high, making TBP's and TNP's especially well suitable for optical tomographic applications. Excitation in the visible region (near 500 nm), featured by regular PtP's and PdP's, is useful in planar wide-field imaging (vide infra), where less diffuse nature of excitation serves to improve spatial resolution. In addition, absorption near 500 nm overlaps with emission of many two-photon (2P) chromophores, which is useful in construction of FRET-enhanced probes for two-photon oxygen microscopy.

TABLE II
Photophysical properties of Pt and Pd complexes of P, TBP and TNP.

| | Absorption | | | |
|---|---|---|---|---|
| | Soret band | Q band | Phosphorescence [a] | |
| Metalloporphyrin | nm (Ig ε) | nm (Ig ε) | λₘₐₓ, nm | φ/τ0, μs [b] |
| Pd-1-OBu | 422 (5.61) | 526 (4.57) | 696 | 0.08/504 |
| Pd-2-OBu | 446 (5.46) | 635 (5.00) | 816 | 0.09/210 |
| Pd-3-OBu | 458 (5.29) | 716 (5.29) | 961 [c] | 0.02 [c]/52 |

[a] Measured using solution in PhCN, deoxygenated by Ar-bubbling.
[b] Emmission quantum yields were determined relative to the flourescence of H₂TPP (φₙ = 0.11) in deoxygenated benzene.
[c] Measurements above 900 nm are subject to large experimental errors due to the very low quantum efficiency of the detection systems in the red part of the spectrum.

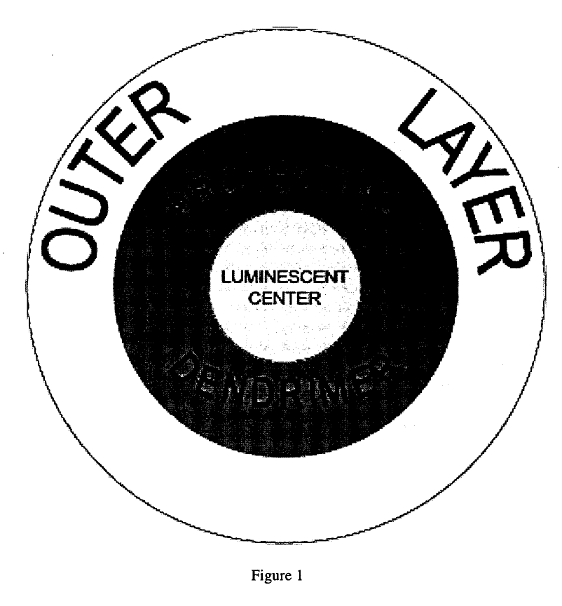

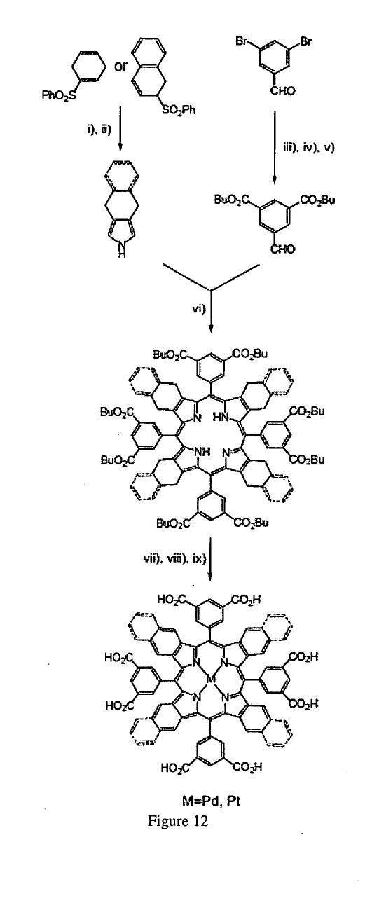

An important feature of porphyrin-based probes is their record-high phosphorescence Stokes shifts achievable via excitation at the Soret bands (e.g. 9329 cm⁻¹ for PdP). Efficient S₂→S₁ internal conversion, [37] combined with extremely high extinction coefficients of S₀—S₂ transitions (˜3×10⁵ M⁻¹ cm⁻¹) makes this pathway superior to the direct S₀—S₁ excitation in those cases when near UV radiation can be sustained by the object. Porphyrins with meso-3,5-dicarboxyphenyl-groups were used in this study as cores for the dendrimers (FIG. 1). Regular tetraarylporphyrins were synthesized by the Lindsey method using 3,5-buthoxycarbonylbenzaldehyde. The same aldehyde was used in the synthesis of π-extended analogues, as depicted in FIG. 12, showing schem 1 (iii-v).

The pathway shown in FIG. 12 is based on the modified Barton-Zard reaction (i), used to generate pyrroles annealed with exocyclic non-aromatic rings, followed by the macrocycle assembly by the Lindsey method (vi), metal insertion into the resulting porphyrins (vii) and oxidative aromatization (viii) into the target π-extended macrocycles. Pt and Pd complexes were obtained in excellent purity and good overall yields. The core porphyrins, also referred below as G0-dendrimers. The footnote to the Scheme contains references to the sources where the corresponding protocols were developed and/or used in similar syntheses.

### Dendritic Cages

Dendritic attenuation of quenching has been documented in a number of studies, where quenchers had different sizes, charges and the quenching processes themselves had different mechanisms (e.g. electron transfer vs energy transfer). If a chromophore is embedded inside a dendrimer, the latter forms a protective cage, preventing physical contacts of the core with macromolecular objects in the environment. However, protecting the chromophore from collisions with small molecules is not as straightforward, since the latter can effectively diffuse through the body of the dendritic matrix.

It is important to realize that quenching constant k_{q} in Eq. 2 is a product of the quencher concentration and the diffusion coefficient, which are both affected by the chromophore environment. If the solubility of oxygen in the solvent (e.g. water) is lower than that in the bulk of the dendrimer, the latter can serve as a “concentrator” or “sink” for oxygen. Still, the decrease in the rate of oxygen diffusion can effectively offset the increase in its local concentration. Hydrophobic dendritic branches fold in polar environments (e.g. water), and as a result their mobility becomes restricted, preventing oxygen molecules from freely reaching the phosphorescent core. Notably, the density of the folded dendrimer may be lower than that of the bulk solvent, but the constrained dynamics of the branches affects the diffusion much more than the density.

Dendrimer dynamics is governed by the interactions of the branches with the solvent. In “good” solvents, the mobility is higher, and oxygen diffusion to the core is attenuated less than in “bad” solvents. Similarly, for the same solvent, dendrimers with more solvent compatible composition limit oxygen access much less than less compatible dendrimers. In particular, dendrimers composed of aromatic motifs are most effective in shielding porphyrins from oxygen in aqueous solutions.

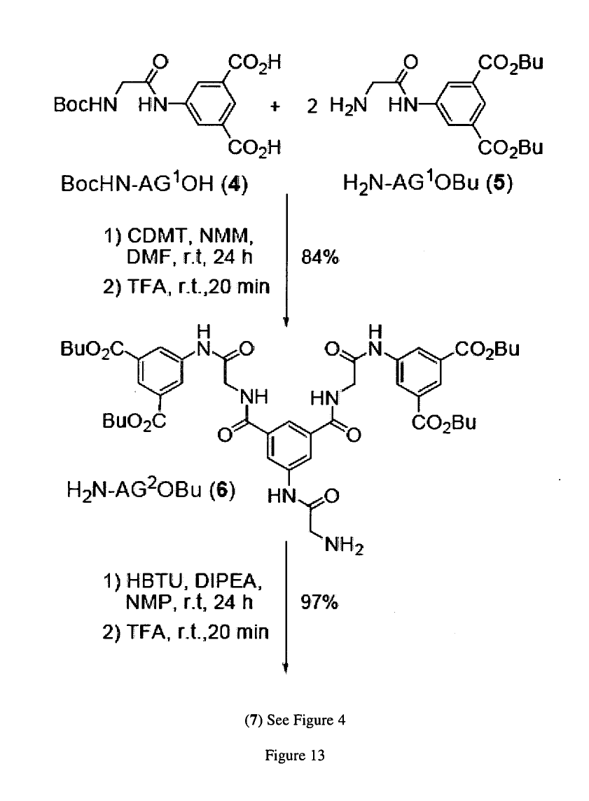

Among many dendrimers with flexible aromatic skeletons, dendritic poly(aryl-glycine) (AG) dendrons (Scheme 2, FIG. 13, FIG. 4) are especially well suited for construction of phosphorescent probes. AG-dendrons offer the advantage of inexpensive starting materials, simplicity of synthesis and chromatography-free purification. Focal amino groups on AG-dendrons complement carboxyls on the core porphyrins, whereas terminal carboxyls on the dendrons provide multiple opportunities for functionalization. AG-dendrons are conveniently functionalized, allowing attachment of cross-linkable groups.

For dendrimers: C-(AGⁿR)ₘ, where C denotes the dendrimer core, AG denotes the dendritic aryl-glycine skeleton, n is the generation number, R is the terminal group and m is the number of dendritic wedges attached to the core. For example, generation 2 AG-dendrimer consisting of PdTBP core and eight AG-dendrons terminated by carboxyl groups is abbreviated as Pt-2-(AG²OH)₈.

The synthesis in Scheme 2 makes use of the Fischer haloacyl halide method to generate building blocks 4 ands 5. The following assembly relies on modern peptide coupling reactions, employing CDMT/NMM and HBTU/DIPEA (see Scheme 2 caption for abbreviations) and permitting synthesis of dendrons 6 and 7 (FIG. 4) in high purity and yield. The AG dendrons can be produced in multigram quantities and stored for long periods of time without detectable decomposition.

### Polyethyleneglycol (PEG) Layer

Special requirements to probes for medical imaging applications include lack of toxicity and excretability from the blood upon completion of imaging. Globular uncharged molecules with molecular weights between 1 and 15 kDa are usually excretable by the kidney. If a probe satisfies this criterion and remains confined to the intravascular space (does not diffuse out of the blood vessels), it is likely to be removed from the blood by the kidney-mediated dialysis, and the possibility of long-term toxicity effects can be avoided.

One way to eliminate interactions of dendritic probes with biological macromolecules and avoid toxicity is to modify their termini with polyethyleneglycol residues. PEGylation of macromolecular compounds for drug delivery and related applications (e.g. artificial blood) is a widely known strategy, including PEGylation of dendrimers. Peripheral polyethyleneglycol groups on porphyrin-dendrimers successfully eliminate interactions of the probes with proteins, while keeping the surface of the probes highly hydrophilic. Although PEG residues themselves also contribute to attenuation of oxygen quenching constants, their effect is small compared to that of the hydrophobic dendritic branches. In addition, the effect of PEG's levels off with an increase in the length of linear chains. As a result, probe molecules of virtually any size can be generated without significantly changing the quenching properties.

### Example 4

### Probe Assembly

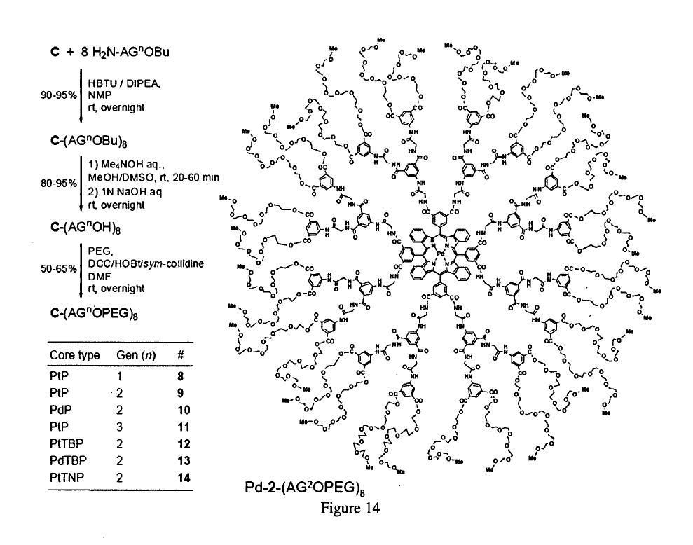

Synthetic assembly of the dendritic probes consists of the attachment of dendrons to the cores, hydrolysis of the peripheral ester groups and modification of the peripheral carboxyls with polyethyleneglycol residues. Each step of this sequence was optimized in order to insure maximal yields and monodispersity of the probes. FIG. 14 shows the complete reaction sequence as well as the structure of a selected probe molecule, Pd-2-(AG²OPEG)₈. The table in the Scheme enumerates the final probe molecules of the general formula C-(AG²OPEG)₈. Using CDMT as a coupling reagent, G1 dendritic branches (5) were attached to the core porphyrins yielding perfect dendrimers C-(AG¹OBu)₈ in 90-95% yields. These dendrimers could be readily purified from excess of 5 by washing the crude mixtures with ethanol. However, CDMT chemistry was not successful in the case of G² and G³ dendrimers. MALDI-TOF analysis of the reaction mixtures revealed presence of imperfect dendrimers with four-to-eight AG²OBu-branches. Additional experiments confirmed that lower molecular weight peaks in the MALDI spectra was not caused by the fragmentation (see Example 1).

Among several well-established peptide-coupling systems (including [(Me₂N)₂CF]⁺PF6⁻, DCC, CDI, uronium-based reagents HBTU (O-benzotriazole-N,N,N′,N′-tetramethyl-uronium hexafluorophosphate) and HATU (2-(¹H-7-Azabenzotriazol-1-yl)-1,1,3,3-tetramethyl uronium hexafluorophosphate), uronium reagents proved to be superior for complete modification of porphyrins with AG²-dendrons 6. However, when used under the originally proposed conditions they still produced mixtures contaminated with imperfect dendrimers. We further established that the single most important parameter with respect to complete derivatization of porphyrins with AG-dendrons is the choice of the solvent. Porphyrin octacarboxylic acids are soluble only in polar aprotic solvents, (e.g. DMF, DMA, NMP, DMSO) and NMP was found to be the best choice. Possibly, porphyrins are much less aggregated in NMP, and NMP contains much fewer free-amine impurities than DMF or DMA. Small amine molecules (e.g. dimethylamine) effectively compete with bulky AG dendrons in the coupling reaction, as evidenced, for example, by the presence of peaks corresponding to C-(AG²OBu)₇NMe₂ in the MALDI spectra of reactions carried out in DMF. Notably, reaction intermediates bearing eight activated carboxyl groups on the porphyrin core are highly unstable at room temperature. Reactants (AG-dendrons) need to be added to the mixtures immediately following the addition of DIPEA.

Complete derivatization of porphyrins required ˜1.5 molar excess of dendron 6. The excess reagent was removed using isothiocyanate-modified resin (Sigma-Aldrich), designed specifically to scavenge molecules with free amino-groups. Thus pre-purified dendrimers were subjected to size-exclusion chromatography (SEC) on SX-1 beads (Biorad) using THF as a mobile phase.

Modification of porphyrins with AG3-dendrons 7 proved to be extremely challenging with a 2:2:1 mixture of dendrimers with six, seven and eight AG3-branches respectively, attached to Pt-1-OH providing operable probes. From the practical point of view, modification of the cores with G2 dendrons provided sufficient attenuation of oxygen sensitivity (vide supra).

In spite of the presence of multiple butyl ester groups, solubility of the porphyrin-AG-dendrimers in common solvents, such as CH₂Cl₂, ether, acetone, methyl and ethyl alcohols, was found to be quite limited; however, AG-dendrimers are well soluble in THF, DMSO and pyridine. Each of the last three solvents is stable in the presence of alkali, permitting hydrolytic cleavage of the terminal esters under basic conditions. However, while attempting to use NaOH in THF/H2O (50:1), MALDI spectra of the reaction mixtures was found to show peaks with lower molecular masses than was expected for dendrimers-polycarboxylic acids, likely due to the partial hydrolysis of anilides in the body of the dendrimer. In order to remove the peripheral butyl groups without affecting the dendrimer integrity, a two-step scheme was devised. At first, poly-(butyl ester) dendrimers were treated with NMe₄OH (˜5 mM) in DMSO/MeOH over 20-60 min period, followed by solvent removal and an subsequent hydrolysis in 0.1N aqueous NaOH overnight. As a result, pure monodisperse dendrimer carboxylic acids could be isolated in 80-95% yields. Importantly, when applied to crude mixtures of C-(AG²OBu)₈ (Scheme 3, FIG. 18), contaminated with unreacted dendrons 6, this two-step sequence yielded completely pure acids C-(AG²OH)₈, containing no traces of dendrons H₂N-AG²OH. From the practical point of view this result is extremely beneficial, as it made possible to avoid chromatographic purification of the butyl-ester dendrimers and thus significantly improve the overall yields. For example, Pt-1-(AG²OH)₈ could be obtained without purification in 81% yield starting from the Pt-1-OH and 6.

At the last stage, the peripheral carboxyl groups on the dendrimers were esterified with monomethoxyoligoethyleneglycol residues (Av. MW 350) in order to obtain water-soluble uncharged probes. Esterification was carried out using the earlier developed DCC/HOBt chemistry. One important practical result of this work is that a convenient work-up procedure after the esterification reaction was developed to entirely avoid chromatographic purification. It was found that by simply re-precipitating PEGylated dendrimers from THF upon addition of diethyl ether pure PEGylated dendrimers could be obtained. The yields of the PEGylation varied in the range of 50-65%. Judged from the MALDI spectra, 95-100% of the carboxyl groups were converted to the PEG-esters.

### Example 5

### Photophysical Properties

Optical properties of the dendrimers in the UV-vis-NIR range are mostly determined by their porphyrin cores (see FIG. 8 for comparison). The absorption bands of all G2 (9, 12-14) and G3 (11) dendrimers in aqueous solutions are very close to those of the parent porphyrins (Pt-1-OBu, Pt-2-OBu and Pt-3-OBu) in PhCN, suggesting that the cores are buried deep inside the dendritic matrix. In contrast, the Soret bands of G1 dendrimer (9) and of the unprotected porphyrin Pt-1-OPEG (8), also in aqueous solutions, are blue-shifted by about 15 nm compared to that of 9.

In addition, the absorption spectrum of G1 dendrimer 8 was found to drift considerably in time. If registered immediately after dissolution, the Soret band revealed a shoulder; however, during about 1 h this shoulder gradually disappeared. Typically, such behavior is associated with porphyrin aggregation in aqueous solutions.

The trends in the emission spectra generally reflect those in the absorption. However, the emission spectra of more aggregated and more solvent-exposed G0 and G1 compounds (Pt-1-OPEG and 8) were found to be shifted to the red, instead of to the blue, relative to the spectrum of the parent porphyrin (Pt-1-OBu) in PhCN. The emission maxima of all probes fall into the tissue NIR window, although phosphorescence of PtTNP-based dendrimer (14) occurs close to its red border. Compared to the Pt porphyrin dendrimers (e.g. 9 and 12), Pd counterparts (10 and 13) exhibit lower quantum yields and significantly longer lifetimes, reflecting the behavior of the parent porphyrins (Tables II and III).

### Example 6

### Phosphorescence Quenching

As shown, the slopes of Stern-Volmer plots decrease significantly with an increase in the dendrimer generation. Overall, between G0 (Pt-1-OPEG) and G3 (11) the quenching rate drops by nearly 20 times.

Having described preferred embodiments of the invention with reference to the accompanying drawings and examples, it is to be understood that the invention is not limited to the precise embodiments, and that various changes and modifications may be effected therein by those skilled in the art without departing from the scope or spirit of the invention as defined in the appended claims.

What is claimed is:

1. A quencher-insensitive nanoprobe comprising: a lumisescent moeity with a long excited state lifetime, embedded in a dendrimer, having a hydrophilic peripheral layer, wherein emission lifetime of the luminescent moiety is longer than the diffusion time of the quencher to the luminescent core.

2. A quencher-insensitive nanoprobe comprising: a lumisescent moeity with a long excited state lifetime, embedded in a dendrimer, wherein emission lifetime of the luminescent moiety is longer than the diffusion time of the quencher to the luminescent core.

3. The nanoprobe of claim 2, wherein said dendrimer has a hydrophilic peripheral layer.

4. The nanoprobe of claim 1 or 2, wherein said luminescent moiety is a luminescent metalloporphyrin represented by any one of the compounds of Formula I-V.

5. The nanoprobe of claim 1 or 3, wherein said peripheral layer is poly(ethyleneglycol) (PEG), poly(lactide-co-glycolide) acid (PLGA), poly(L-lactide) acid (PLLA), poly(D-lactide) acid (PDLA), is polyvinylalcohol (PVOH) or polysorbate.

6. The nanoprobe of claim 1 or 2 wherein said dendrimer is internally cross-linked to prevent diffusional quenching by oxygen.

7. The nanoprobe of claim 1 or 3, wherein said polymeric layer is optionally connected to an agent capable of binding to a pre-determined target.

8. The nanoprobe of claim 1 or 3, having the general formula:
C^{π}-(AGⁿ-R)ₘ
where:
C is a metalloporphirine core;
π is the number of π-extensions of the metalloporphirine core;
AG is the dendritic aryl-glycine skeleton;
n is the generation number;
R is the peripheral unit; and
m is the number of dendritic wedges attached to the core.

9. The nanoprobe of claim 8, wherein said metal of said metalloporphirin is palladium or platinum ions.

10. The nanoprobe of claim 8, wherein said n is between 1 and 5.

11. The nanoprobe of claim 8, wherein said m is between 6 and 8.

12. The nanoprobe of claim 8, wherein π is between 0 and 3.

13. The nanoprobe of claim 2, wherein the dendrimer has a hydroxyl terminal group.

14. A method of providing imaging contrast to a tissue of a subject, comprising the step of administering to the tissue the nanoprobe of claim 1; exposing the tissue to an electromagnetic radiation, thereby exciting the nanoprobe; and using an imaging device, imaging the tissue.

15. The method of claim 14, whereby excitation is in a form of a light pulse, which is shorter than, or equals to the luminescence lifetime of the luminescent moiety and the step of imaging is performed after a delay following the excitation pulse, but prior to the end of the luminescence decay, thus eliminating the background signal and increasing the signal-to-noise ratio.

16. A method for an in-vivo imaging of a tumor neovasculature in a subject comprising (i) administering a quencher-insensitive nanoprobe comprising: a lumisescent moeity with a long excited m state lifetime, embedded in a dendrimer, optionally having a hydrophilic peripheral layer, wherein emission lifetime of the luminescent moiety is longer than the diffusion time of the quencher to the luminescent core; (ii) exciting said luminescent moeity; (iii) detecting light emitted from said tumor neovasculature.

17. An optical imaging system comprising:
an electronic imaging device configured to capture an image of a predetermined site;
a quencher-insensitive nanoprobe comprising: a lumisescent moeity with a long excited state lifetime, embedded in a dendrimer with hydrophilic peripheral layer, wherein said dendrimer isolates the chromophore from the measurement environment and eliminates unwanted quenching by posing a kinetic barrier to the quenching species; and
a projector configured to project a visible representation of the captured image.

18. The optical imaging system of claim 17, wherein the predetermined site is a tumor, a lesion, a digestive tract, a lymph node, a brain tissue, a lung tissue or a nervous system tissue.

19. The optical imaging system of claim 18, further comprising an excitation light source capable of providing one or more wavelengths to excite the nanoprobe.

20. The optical imaging system of claim 17, wherein said luminescent moiety is a luminescent moiety is a luminescent metalloporphyrin represented by any one of the compounds of Formula I-V.

21. The optical imaging system of claim 17, wherein said peripheral layer is poly(ethyleneglycol) (PEG), poly(lactide-co-glycolide) acid (PLGA), poly(L-lactide) acid (PLLA), poly(D-lactide) acid (PDLA), polyvinylalcohol (PVOH) or polysorbate.

22. The optical imaging system of claim 17, wherein said hydrophilic peripheral layer is optionally connected to an agent capable of binding to a pre-determined target.

23. The optical imaging system of claim 17, wherein said nanoprobe has the general formula:
C^{π}-(AGⁿ-R)ₘ
where:
C is a metalloporphirine core;
π is the number of n-extensions of the metalloporphirine core
AG is the dendritic aryl-glycine skeleton;
n is the generation number;
R is the peripheral unit; and
m is the number of dendritic wedges attached to the core.

24. The optical imaging system of claim 23, wherein said metal of said metalloporphirine is m palladium or platinum ions.

25. The optical imaging system of claim 23, wherein said n is between 1 and 5.

26. The optical imaging system of claim 23, wherein said m is between 6 and 8.

27. The optical imaging system of claim 23, wherein π is between 0 and 3.

28. The optical imaging system of claim 23, wherein R is a polyethylene glycol unit.

29. The optical imaging system of claim 23, wherein the excitation light source is capable of providing light wavelength in the range of between about 410 and 960 nm.

30. The optical imaging system of claim 23, wherein said dendrimer is internally cross-linked to prevent diffusional quenching by oxygen.

31. The optical imaging system of claim 23, wherein the dendrimer has a hydroxyl terminal group, without a peripheral polymeric layer.
